SEQUENCE LISTING (40 sequences)

SEQ ID NO: 1 - protein, 109 aa, Artificial sequence
  antigen-binding domain
EIVLTQSPATLSLSPGERATLSCSASSSVSYMQWYQQKPGLAPRLLIYDTSKLTSGIPDRFSGSGSGTDFTLTISRLEPEDFAVYYCQQWSSNPLTFGGGTKVEIKRTV

SEQ ID NO: 2 - protein, 123 aa, Artificial sequence
  antigen-binding domain
EVQLVQSGAEVKKPGASVKVSCKASGYTFTEYTMHWVRQAPGQGLEWIGGINPNTGGTTYNQKFNGRVTMTRDTSISTAYMELSSLRSEDTAVYYCTRPYRDDGGFHWYFDVWGQGTLVTVSS

SEQ ID NO: 3 - protein, 110 aa, Artificial sequence
  antigen-binding domain
DIQLTQSPSFLSASVGDRVTINCRASKTISNYLAWYQQKPGKAPKLLIYSGSTLQSGVPSRFSGSGSGTDFTLTISSLQPEDFATYYCQQHHEYPLTFGGGTKVEIKRTV

SEQ ID NO: 4 - protein, 122 aa, Artificial sequence
  antigen-binding domain
QVQLVQSGAEVKKPGASVKVSCKVSGYTFTDGAMHWVRQAPGQGLEWIGIINTNSGNTNYNQKFQGRVTMTRDTSISTAYMELSRLRSEDTAVYYCARGVFYYGYGAWFAYWGQGTLVTVSS

SEQ ID NO: 5 - protein, 106 aa, Artificial sequence
  antigen-binding domain
QIVLTQSPAVMSTSPGEKVTMTCSASSSVSYMQWYQQKSGTSPKRWIYDTSKLTSGVPARFSGSGSGTSYSLTISSMEAEDAATYYCQQWSSNPLTFGAGTKLELK

SEQ ID NO: 6 - protein, 123 aa, Artificial sequence
  antigen-binding domain
EVQLQQSGPELVKPGASVKISCKTSGYAFTEYTMHWVKQSQGKSLEWIGGINPNTGGTTYNQKFNGKATLTVDRSSSTAYMELRSLTSEDSAVYYCTRPYRDDGGFHWYFDVWGAGTAVTVSS

SEQ ID NO: 7 - protein, 107 aa, Artificial sequence
  antigen-binding domain
DVQITQSPSYLTASPGETIIINCRASKTISNYLAWYQEKPGKTNKLLIYSGSTLQSGIPSRFSGSGSDTDFTLTISSLEPEDFAMYYCQQHHEYPLTFGAGTKLELK

SEQ ID NO: 8 - protein, 122 aa, Artificial sequence
  antigen-binding domain
QVQLQQSGPELVRPGVSVKISCKVSGYTFTDGAMHWVKRSHAKSLEWIGIINTNSGNTNYNQKFQGKATMTVDKSSSTAYMELARLTSEDSAIYYCARGVFYYGYGAWFAYWGQGTLVTVSA

SEQ ID NO: 9 - protein, 113 aa, Artificial sequence
  antigen-binding domain
DIVMTQSPSSLTVTAGENVTMSCKSSQTLLNNGNQKNFLTWYQQKPGQPPKLLIYLASTRESGVPDRFTGSGSGTDFTLTISSVQAEDLAVYYCQNDYTYPLTFGAGTKLELK

SEQ ID NO: 10 - protein, 120 aa, Artificial sequence
  antigen-binding domain
EVKLVESGGGLVQPGGSLRLSCATSGFTFTDYYMSWVRQPPGKALEWLGFIRNKVNDYTTEYSVSVKGRFTISRDNSQTILYLQMNTLRAEDSATYYCARDSPYRPFAYWGQGTLVTVSA

SEQ ID NO: 11 - protein, 113 aa, Artificial sequence
  antigen-binding domain
DIQMTQSPSSLTVTAGEKVTMSCKSSQSLLNSGNQNNYLTWYQQKPGQPPKLLIYLASTRDSGVPDRFTGSGSGTDFTLTISSVQAEDLAVYYCQNDYTYPLTFGAGTKLELK

SEQ ID NO: 12 - protein, 120 aa, Artificial sequence
  antigen-binding domain
EVKLVESGGGLVQPGGSLRLSCATSGFTFTDYYMSWVRQPPGKALEWLGFIRNKANGYTTEYSASVKGRFTISSDDSQSILYLQMNTLRAEDSATYYCARDSHYRPFAYWGQGTLVTVSA

SEQ ID NO: 13 - protein, 107 aa, Artificial sequence
  antigen-binding domain
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 14 - protein, 122 aa, Artificial sequence
  antigen-binding domain
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 15 - protein, 106 aa, Artificial sequence
  antigen-binding domain
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 16 - protein, 122 aa, Artificial sequence
  antigen-binding domain
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 17 - protein, 492 aa, Artificial sequence
  anti-B7H3 chimeric antigen receptor H26B6-CAR
MDMRVPAQLLGLLLLWLRGARCEIVLTQSPATLSLSPGERATLSCSASSSVSYMQWYQQKPGLAPRLLIYDTSKLTSGIPDRFSGSGSGTDFTLTISRLEPEDFAVYYCQQWSSNPLTFGGGTKVEIKRTVGGGGSGGGGSGGGGSEVQLVQSGAEVKKPGASVKVSCKASGYTFTEYTMHWVRQAPGQGLEWIGGINPNTGGTTYNQKFNGRVTMTRDTSISTAYMELSSLRSEDTAVYYCTRPYRDDGGFHWYFDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 492 aa, Artificial sequence
  anti-B7H3 chimeric antigen receptor H2B8-CAR
MDMRVPAQLLGLLLLWLRGARCDIQLTQSPSFLSASVGDRVTINCRASKTISNYLAWYQQKPGKAPKLLIYSGSTLQSGVPSRFSGSGSGTDFTLTISSLQPEDFATYYCQQHHEYPLTFGGGTKVEIKRTVGGGGSGGGGSGGGGSQVQLVQSGAEVKKPGASVKVSCKVSGYTFTDGAMHWVRQAPGQGLEWIGIINTNSGNTNYNQKFQGRVTMTRDTSISTAYMELSRLRSEDTAVYYCARGVFYYGYGAWFAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 487 aa, Artificial sequence
  anti-B7H3 chimeric antigen receptor L26B6-CAR
METPAQLLFLLLLWLPDTTGQIVLTQSPAVMSTSPGEKVTMTCSASSSVSYMQWYQQKSGTSPKRWIYDTSKLTSGVPARFSGSGSGTSYSLTISSMEAEDAATYYCQQWSSNPLTFGAGTKLELKGGGGSGGGGSGGGGSEVQLQQSGPELVKPGASVKISCKTSGYAFTEYTMHWVKQSQGKSLEWIGGINPNTGGTTYNQKFNGKATLTVDRSSSTAYMELRSLTSEDSAVYYCTRPYRDDGGFHWYFDVWGAGTAVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 488 aa, Artificial sequence
  anti-B7H3 chimeric antigen receptor 26B6-CAR
MALPVTALLLPLALLLHAARPQIVLTQSPAVMSTSPGEKVTMTCSASSSVSYMQWYQQKSGTSPKRWIYDTSKLTSGVPARFSGSGSGTSYSLTISSMEAEDAATYYCQQWSSNPLTFGAGTKLELKGGGGSGGGGSGGGGSEVQLQQSGPELVKPGASVKISCKTSGYAFTEYTMHWVKQSQGKSLEWIGGINPNTGGTTYNQKFNGKATLTVDRSSSTAYMELRSLTSEDSAVYYCTRPYRDDGGFHWYFDVWGAGTAVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 489 aa, Artificial sequence
  anti-B7H3 chimeric antigen receptor L2B8-CAR
MDMRVPAQLLGLLLLWLRGARCDVQITQSPSYLTASPGETIIINCRASKTISNYLAWYQEKPGKTNKLLIYSGSTLQSGIPSRFSGSGSDTDFTLTISSLEPEDFAMYYCQQHHEYPLTFGAGTKLELKGGGGSGGGGSGGGGSQVQLQQSGPELVRPGVSVKISCKVSGYTFTDGAMHWVKRSHAKSLEWIGIINTNSGNTNYNQKFQGKATMTVDKSSSTAYMELARLTSEDSAIYYCARGVFYYGYGAWFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 488 aa, Artificial sequence
  anti-B7H3 chimeric antigen receptor 2B8-CAR
MALPVTALLLPLALLLHAARPDVQITQSPSYLTASPGETIIINCRASKTISNYLAWYQEKPGKTNKLLIYSGSTLQSGIPSRFSGSGSDTDFTLTISSLEPEDFAMYYCQQHHEYPLTFGAGTKLELKGGGGSGGGGSGGGGSQVQLQQSGPELVRPGVSVKISCKVSGYTFTDGAMHWVKRSHAKSLEWIGIINTNSGNTNYNQKFQGKATMTVDKSSSTAYMELARLTSEDSAIYYCARGVFYYGYGAWFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 493 aa, Artificial sequence
  anti-B7H3 chimeric antigen receptor L23H1-CAR
MDMRVPAQLLGLLLLWLRGARCDIVMTQSPSSLTVTAGENVTMSCKSSQTLLNNGNQKNFLTWYQQKPGQPPKLLIYLASTRESGVPDRFTGSGSGTDFTLTISSVQAEDLAVYYCQNDYTYPLTFGAGTKLELKGGGGSGGGGSGGGGSEVKLVESGGGLVQPGGSLRLSCATSGFTFTDYYMSWVRQPPGKALEWLGFIRNKVNDYTTEYSVSVKGRFTISRDNSQTILYLQMNTLRAEDSATYYCARDSPYRPFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 492 aa, Artificial sequence
  anti-B7H3 chimeric antigen receptor 23H1-CAR
MALPVTALLLPLALLLHAARPDIVMTQSPSSLTVTAGENVTMSCKSSQTLLNNGNQKNFLTWYQQKPGQPPKLLIYLASTRESGVPDRFTGSGSGTDFTLTISSVQAEDLAVYYCQNDYTYPLTFGAGTKLELKGGGGSGGGGSGGGGSEVKLVESGGGLVQPGGSLRLSCATSGFTFTDYYMSWVRQPPGKALEWLGFIRNKVNDYTTEYSVSVKGRFTISRDNSQTILYLQMNTLRAEDSATYYCARDSPYRPFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 493 aa, Artificial sequence
  anti-B7H3 chimeric antigen receptor L6F7-CAR
MDMRVPAQLLGLLLLWLRGARCDIQMTQSPSSLTVTAGEKVTMSCKSSQSLLNSGNQNNYLTWYQQKPGQPPKLLIYLASTRDSGVPDRFTGSGSGTDFTLTISSVQAEDLAVYYCQNDYTYPLTFGAGTKLELKGGGGSGGGGSGGGGSEVKLVESGGGLVQPGGSLRLSCATSGFTFTDYYMSWVRQPPGKALEWLGFIRNKANGYTTEYSASVKGRFTISSDDSQSILYLQMNTLRAEDSATYYCARDSHYRPFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 492 aa, Artificial sequence
  anti-B7H3 chimeric antigen receptor 6F7-CAR
MALPVTALLLPLALLLHAARPDIQMTQSPSSLTVTAGEKVTMSCKSSQSLLNSGNQNNYLTWYQQKPGQPPKLLIYLASTRDSGVPDRFTGSGSGTDFTLTISSVQAEDLAVYYCQNDYTYPLTFGAGTKLELKGGGGSGGGGSGGGGSEVKLVESGGGLVQPGGSLRLSCATSGFTFTDYYMSWVRQPPGKALEWLGFIRNKANGYTTEYSASVKGRFTISSDDSQSILYLQMNTLRAEDSATYYCARDSHYRPFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 488 aa, Artificial sequence
  anti-B7H3 chimeric antigen receptor Eno-CAR
MALPVTALLLPLALLLHAARPDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKGGGGSGGGGSGGGGSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 487 aa, Artificial sequence
  anti-B7H3 chimeric antigen receptor huM30-CAR
MALPVTALLLPLALLLHAARPEIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKGGGGSGGGGSGGGGSQVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 1479 bp, Artificial sequence
  coding gene of H26B8-CAR
atggatatgcgcgttcctgctcaactgctgggactgctcctcctgtggctgagaggcgccaggtgtgagatcgttctgacccagtctcccgcaaccctgtctctcagccctggcgagcgcgctactctgagctgcagcgcttcctcatctgtgagttatatgcagtggtatcagcaaaagcccggcctggctccacgcctcctcatatacgatacttccaaactcacttccggtattcccgatcggttctccggctccggcagtggcactgactttaccctcaccatcagtcgcctggagcctgaagattttgcagtgtactactgccaacagtggagctctaacccactgacctttggcggaggaacaaaagttgaaattaagaggactgtcggcggtggcggtagtggaggcggcggatctggaggtggtggtagcgaggtgcagctggtgcagagtggcgccgaggtgaagaagcctggcgcatccgtcaaggtgagctgtaaggcaagcgggtacacctttacagagtataccatgcattgggttcggcaggctcccggccagggcctcgaatggatcggtgggatcaaccctaacacaggtggaactacatacaaccagaagttcaatggacgggtgaccatgactcgggatacatccatcagcaccgcatatatggaactgtcaagcctgcgctctgaggacactgccgtctactactgtacaaggccatacagagacgacggaggatttcactggtactttgacgtgtgggggcagggcaccctcgttaccgttagctcaactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 30 - DNA, 1479 bp, Artificial sequence
  coding gene of H2B8-CAR
atggatatgcgcgttcctgctcaactcctcggtctgctcctgctgtggctcaggggcgccaggtgtgacatccagctgacccagtcaccatccttcctgagcgcctccgtgggcgacagagttactatcaattgccgggcctcaaagactatatctaattatctggcttggtatcagcagaagccagggaaggcaccaaagctcctgatctactccggctccactctgcagtccggcgtgcccagtagattttctggcagcgggtcagggaccgacttcaccctcacaattagtagtctgcagccagaggatttcgccacttattactgccaacagcatcatgaatatccactgacctttggtggcggaaccaaggtcgagatcaagcggacagttggtggaggaggcagtggcggaggcggcagtggaggcggaggcagtcaagtccaactggtgcagtctggcgcagaagtgaagaagccaggcgccagtgttaaggtgtcctgtaaagtcagcggctatacctttacagacggtgcaatgcattgggtcagacaggcaccagggcagggtctggagtggatcgggatcatcaatacaaacagcggaaacactaattacaaccagaaattccagggaagagtgactatgactagggacacatcaatctcaaccgcatatatggaactgagcaggctgcgctccgaggacaccgccgtctactactgtgcacgcggcgtgttttactatggatacggagcatggttcgcttactggggccaagggactctggttacagtctcttcaactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 31 - DNA, 1464 bp, Artificial sequence
  coding gene of L26B6-CAR
atggagacacctgcccagctgctgttcctgctgctgctgtggctgcccgacaccacaggacagatcgtgctgacccagtctcctgccgtgatgtccacctctcctggcgagaaggtgaccatgacctgctctgcctcctcctccgtgtcctacatgcagtggtaccagcagaagtccggcacctctcccaagcggtggatctacgacacctccaagctgacctctggcgtgcctgctcggttctccggatctggctctggcacctcctactccctgaccatctcctccatggaggccgaggacgctgccacctactactgccagcagtggtcctccaaccctctgaccttcggagctggcaccaagctggagctgaagggtggaggagggtctggtggtggaggcagcggaggcggtggctctgaggtgcagctgcagcagtctggacccgagctggtgaagcctggagcctccgtgaagatctcctgcaagacctccggctacgccttcaccgagtacaccatgcactgggtgaagcagtcccagggcaagtccctggagtggatcggaggcatcaaccccaacacaggaggcaccacctacaaccagaagttcaacggcaaggccaccctgaccgtggaccggtcctcctccaccgcctacatggagctgcggtccctgacctccgaggactccgccgtgtactactgcaccagaccctaccgggacgacggaggcttccactggtacttcgacgtgtggggagctggcacagccgtgaccgtgtcctccactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 32 - DNA, 1467 bp, Artificial sequence
  coding gene of 26B6-CAR
atggcactgcccgtcacagcactgctcctcccactcgctctgctcctgcacgctgctaggcctcagatagtgctgacacagagtcccgctgtgatgagcacatctccaggcgagaaggtcaccatgacctgcagtgccagcagttcagtgtcatatatgcaatggtaccagcaaaagagcgggaccagtcctaagcgctggatctacgatacaagcaagctgacatcaggagtcccagcaaggttctctgggtccggttccgggacatcctatagcctgactatttcaagcatggaggcagaagatgccgctacctactactgccagcagtggagtagcaatccactgacctttggagccgggaccaagctggagctcaagggaggaggcgggagcggtggcggaggtagcggcggaggcgggtccgaagttcagctgcagcagtctggacccgagctggttaaacctggagcaagcgtgaagatctcatgtaaaaccagtgggtacgcctttactgaatacactatgcactgggtgaaacaaagccaagggaaaagtctggagtggattggcgggatcaatcccaatacaggcggaaccacttacaatcagaagttcaatgggaaggccaccctgactgtcgatagaagtagctccactgcatatatggaactccgctcactcacatccgaggactcagctgtttattactgcacaagaccttatagagacgatggaggctttcattggtatttcgacgtctggggtgctggcaccgctgtcacagtgagcagcactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 33 - DNA, 1470 bp, Artificial sequence
  coding gene of L2B8-CAR
atggacatgcgggtgcctgctcagctgctgggcctgctgctgctgtggctgagaggagccaggtgcgacgtgcaaattactcagtcaccttcatatctcacagcatcaccaggtgaaaccatcatcattaattgtcgcgccagcaaaacaatttctaactatctggcatggtatcaggaaaagcctggaaagactaacaagctcctcatctatagtggctctaccctccagagtggtatcccatctcgcttcagcggcagcggatcagatactgatttcacactgacaatatccagcctggaacccgaggatttcgcaatgtattactgccagcagcatcacgagtaccctctgaccttcggcgcaggcactaaactggagctcaagggaggaggaggctccggtggaggagggagcggcggaggaggcagccaggtccagctgcaacagtcaggccctgaactcgttcggccaggcgtctcagtgaagatctcctgcaaggtgtcaggttacactttcaccgacggcgctatgcattgggtgaagcggtcacacgcaaagtcactggagtggattggaatcatcaataccaactccggtaataccaattataaccagaagttccagggcaaggccactatgacagtggacaagagttcaagtaccgcatacatggagctggctcgcctcacatcagaggactctgcaatctattactgtgctcgcggcgtgttctattatggttatggcgcatggtttgcatactggggccagggtaccctcgttaccgtgtccgccactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 34 - DNA, 1467 bp, Artificial sequence
  coding gene of 2B8-CAR
atggctctgccagttacagcactcctcctccctctcgcactcctgctccatgcagccagacccgacgtgcaaataacccaaagtccctcctacctcactgcttcacctggagaaacaattatcattaactgtcgcgcctctaagacaatctctaattacctggcctggtatcaagagaaacccggcaagactaataaactgctgatctactctgggagtaccctgcaatccggtattccttcccgcttctccggctccgggagtgataccgactttacactgacaattagcagtctggagcccgaggactttgcaatgtactattgccagcaacatcacgagtatccactgaccttcggagccggcaccaagctggaactcaaaggcggaggaggaagtggcggcggtggttctggaggaggaggatctcaggttcagctgcagcagagtggaccagaactcgtgcgcccaggcgtgagcgtgaagatcagctgtaaagtgtccggctatactttcactgacggagccatgcactgggtcaagaggagccacgcaaaatccctggagtggattggcattatcaatacaaatagcggaaatacaaactataaccagaaattccagggcaaagcaacaatgaccgtcgacaagagctccagcacagcttacatggagctggctaggctcaccagcgaggactcagccatatattactgcgctagaggagtgttctattacggctatggtgcttggtttgcctactgggggcaaggaactctcgtcaccgtgagcgccactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 35 - DNA, 1482 bp, Artificial sequence
  coding gene of L23H1-CAR
atggacatgcgggtgcctgctcagctgctgggcctgctgctgctgtggctgagaggagccaggtgcgatattgttatgacccaaagtccatcctctctgacagtgacagctggcgaaaacgttaccatgagttgcaagtcaagtcagaccctgctcaacaacggtaatcagaagaatttcctgacctggtaccagcagaagcccggacagccacccaaactgctgatctacctggcttccactcgcgagagcggagtgcccgataggttcaccgggtcaggcagcggaaccgacttcacactgacaatatcatctgtgcaggcagaggatctcgccgtttattactgccagaatgactatacatacccactcacatttggcgccggaactaagctggagctgaagggaggaggcgggtccggcggaggcggttcaggaggcggcggaagtgaagtgaaactggttgaatctggaggcggtctggtgcaacccggaggctccctgagactcagctgcgccacctccgggttcacttttacagactattatatgagttgggtccgccagccaccaggcaaagccctggaatggctcggctttattagaaataaggtgaacgattacactactgaatatagcgtgtccgttaaagggcgctttaccatcagccgggataatagccagacaatactgtacctgcaaatgaataccctcagagccgaggatagcgctacatactactgtgctcgggactcaccatacaggcccttcgcttattggggacagggtactctggtcacagtttctgcaactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 36 - DNA, 1479 bp, Artificial sequence
  coding gene of 23H1-CAR
atggctctgcctgttactgctctgctgctgcctctggctctcctgctgcatgctgccaggccagatatcgtcatgacccaatctccatcatcactcaccgtcacagctggagaaaatgtgaccatgtcatgcaaaagtagtcagaccctgctcaataatggtaaccagaagaactttctcacttggtatcagcagaaacccgggcagccaccaaagctcctgatctacctcgccagtacacgggaatcaggtgtgcccgataggttcaccgggagtggttccggcacagacttcaccctgacaatcagcagtgtgcaggccgaggacctggccgtttactattgccagaacgattatacctatcctctgacttttggcgctgggacaaaactggagctgaagggaggtggcgggtcaggaggaggcgggtctggcggcggaggttctgaggtgaaactggtggaatcaggtggcggcctggtgcagcctggcggatctctgaggctgagttgcgccaccagtgggtttactttcacagattattacatgtcttgggtgaggcagccacctggaaaggccctggaatggctcggcttcatccggaacaaagtgaatgactacaccacagagtacagtgtgagcgtgaagggcagattcaccatctcccgggataacagtcagaccatcctgtatctgcagatgaacaccctgcgggctgaggattcagcaacctactactgtgcaagagacagcccttatcggcccttcgcctactggggacagggaacactggtgaccgtctctgcaactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 37 - DNA, 1482 bp, Artificial sequence
  coding gene of L6F7-CAR
atggacatgcgggtgcctgctcagctgctgggcctgctgctgctgtggctgagaggagccaggtgcgacatccagatgacccagtctccctcctccctgaccgtgacagctggcgagaaggtgaccatgtcctgcaagtcctcccagtccctgctgaactccggcaaccagaacaactacctgacctggtaccagcagaagcctggccagcctcccaagctgctgatctacctggcctccaccagagactccggcgtgcccgaccggttcaccggatctggctctggcaccgacttcaccctgaccatctcctccgtgcaggccgaggacctggccgtgtactactgccagaacgactacacctaccctctgaccttcggagctggcaccaagctggagctgaagggtggaggagggtctggtggtggaggcagcggaggcggtggctctgaggtgaagctggtggagtctggaggaggcctggtgcagcctggaggctccctgaggctgtcctgcgccacctctggcttcaccttcaccgactactacatgtcctgggtgaggcagcctcctggcaaggccctggagtggctgggcttcatccggaacaaggccaacggctacaccaccgagtactctgcctccgtgaagggacggttcaccatctcctccgacgactcccagtccatcctgtacctgcagatgaacaccctgagagctgaggactccgccacctactactgcgccagagactcccactacagacccttcgcctactggggacagggcaccctggtgaccgtgtctgccactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 38 - DNA, 1479 bp, Artificial sequence
  coding gene of 6F7-CAR
atggccctcccagtgacagccctgctgctgcctctcgctctgctcctccatgctgcaagaccagacatacagatgacacagtctccttcatcactgaccgtgactgccggcgagaaagtcactatgtcttgcaaaagtagccagtctctcctgaacagtgggaaccaaaacaattacctgacatggtatcagcaaaagcccgggcagcctcccaagctcctgatctacctggcttccacccgggacagcggagtgcccgaccggtttactgggagtggctctggaaccgacttcacactgaccatatcctccgtgcaggctgaagacctggctgtgtattattgccaaaatgattatacttatccactgacctttggtgccggtacaaaactggagctgaaaggaggcggagggtcaggtggcggaggctcaggcggaggcggctcagaggtcaagctcgtcgagagcggcggaggactggtgcaacccggtggctcactgcgcctgagctgcgccacctctgggttcactttcaccgactattacatgtcatgggttaggcagcctcctgggaaggccctggagtggctgggcttcatccggaataaagctaacggatataccactgaatacagcgcaagtgtcaagggccgctttaccatttcttccgacgattctcagtctatactctatctgcagatgaacactctgagagccgaagatagcgcaacctactactgcgctagggacagccactaccggccctttgcctattggggtcaaggcaccctcgttaccgtctctgctactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

SEQ ID NO: 39 - DNA, 1464 bp, Artificial sequence
  coding gene of Eno-CAR
atggcactgcctgtgactgccctcctgctgcctctggcactcctgctccacgcagcccggcctgacatccagctgactcagtcaccctctttcctgagcgcatctgtgggagacagggttaccatcacctgcaaggcaagccaaaatgtggacaccaacgtggcctggtatcagcagaagccaggcaaggcacccaaagccctgatctacagcgccagctaccgctactccggagtcccatctcggttctctggatcaggcagcggaaccgactttacactgacaatctcaagcctccaaccagaggactttgccacctattactgccagcagtacaacaattaccctttcacattcgggcagggaaccaagctggaaattaaaggcggcggtggatctggaggtggcgggagtggtggaggagggtcagaggtgcagctggtggagtctggtggtggactggtccaaccaggcggttctctgcgcctcagttgtgccgcctcagggtttacattctctagcttcggaatgcattgggtgaggcaagctccaggcaaaggtctggaatgggtggcttacatctcttccgacagttcagccatctattacgctgataccgttaagggccgctttaccatcagcagagataatgccaagaactcactgtacctgcagatgaatagtctccgggatgaggacaccgcagtctattattgcggtagaggtcgggagaatatatactacggctccagactggactactggggccaagggactactgtcaccgtgagctccactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaagg

SEQ ID NO: 40 - DNA, 1464 bp, Artificial sequence
  coding gene of huM30-CAR
atggctctgcctgttactgctctgctcctgcctctggctctgctgctgcacgccgcacggcccgagatcgtcctgacacagagtcctgccaccctcagcctcagtcctggcgaaagagccaccctgtcctgtagagcaagctcaagactgatatacatgcactggtaccaacagaaacccggacaggcaccaagacctctgatatatgccacaagcaacctggcatcagggataccagctagattttctggatctggtagcggcactgatttcaccctcactatctccagtctggagcctgaagatttcgctgtgtactactgccagcaatggaactctaacccacccactttcgggcagggcacaaaggtcgagattaaaggtggaggcgggtcaggaggaggaggatctggaggtggcgggagtcaggtccagctggtccagtccggcgctgaagttaagaaaccaggtagttctgttaaagtctcatgcaaggccagcggctacacttttacaaattatgtcatgcactgggtgcggcaggctccaggacaagggctcgagtggatgggctatattaacccttataatgacgatgtgaagtataatgagaaattcaaagggagggtgaccatcactgccgacgagagtacctcaaccgcatacatggagctgtcctccctgaggtccgaggacaccgccgtgtactattgcgcacgctgggggtattatgggagcccactgtactactttgactactggggacagggtaccctcgtcaccgtgtcctccactacgacccctgcaccgcggccgcctactcctgcacctacaatcgcaagtcagccactgagtctcagacccgaagcatgccgccctgctgcaggcggagctgtccatacacgcggactggactttgcatgcgatatatacatctgggcaccactggccggcacttgcggcgtgctgctcctgtccctcgtgattaccctgtactgcaaacgcggcaggaagaagctcctgtatatctttaaacagcccttcatgaggccagtgcagaccactcaagaggaagacggttgtagctgccggtttcccgaggaagaagagggaggctgcgagctccgcgtgaagttctcccgctcagccgatgcacccgcctatcagcaagggcagaaccagctgtacaatgagctcaacctgggaagaagggaggaatatgacgttctggataaacggcgcggtcgcgatcccgaaatgggtgggaagcctcgcaggaagaatcctcaggaagggctctacaatgagctgcagaaagacaaaatggcagaggcctattctgaaatcggcatgaagggcgagcgccgcagaggcaaaggacacgacggcctgtaccagggcctgtctacagccaccaaggacacctatgacgctctccacatgcaagccctgccaccaaggtga

(19) United States
(12) Patent Application Publication
(10) Pub. No.: US 2024/0059776 A1
(43) Pub. Date: Feb. 22, 2024

(54) ANTI-B7H3 CHIMERIC ANTIGEN RECEPTOR AND APPLICATION THEREOF

(71) Applicant: GUANGZHOU BIO-GENE TECHNOLOGY CO., LTD, Guangdong (CN)

(72) Inventors: Guangchao Li, Guangdong (CN); Min Luo, Guangdong (CN); Wen Ding, Guangdong (CN); Zhao Zhou, Guangdong (CN); Xuejun Wang, Guangdong (CN)

(73) Assignee: GUANGZHOU BIO-GENE TECHNOLOGY CO., LTD, Guangdong (CN)

(21) Appl. No.: 18/267,295

(22) PCT Filed: Dec. 22, 2020
(86) PCT No.: PCT/CN2020/138243

§ 371 Date: Jun. 14, 2023

(30) Foreign Application Priority Data
Dec. 14, 2020 (CN) 202011476183.6

(51) Int. Cl. C07K 16/28; C07K 14/705; C07K 14/725; C12N 5/0783; A61K 39/00; A61P 35/00

(52) U.S. Cl. CPC C07K 16/2827; C07K 14/70517; C07K 14/70521; C07K 14/7051; C12N 5/0636; A61K 39/464411; A61K 39/4631; A61K 39/4611; A61P 35/00; C07K 2319/03; C07K 2319/02; C07K 2317/24

(57) ABSTRACT

Provided are an anti-B7H3 chimeric antigen receptor and an application thereof. The anti-B7H3 chimeric antigen receptor comprises an antigen binding domain, a hinge region, a transmembrane domain and a signaling transfer structural domain. The antigen binding domain is an anti-human B7H3 antibody. The anti-B7H3 chimeric antigen receptor has a specific targeting effect on B7H3-positive tumor cells, T cells that express the anti-B7H3 chimeric antigen receptor have significant killing effects in vitro and in vivo, can effectively remove the B7H3-positive tumor cells, and having important significance in the field of tumor therapy.

### CROSS-REFERENCE TO RELATED APPLICATIONS

This is a National Stage Application filed under 35 U.S.C. 371 based on International Patent Application No. PCT/CN2020/138243, filed on Dec. 22, 2020, which claims priority to Chinese Patent Application No. 202011476183.6, filed with the China National Intellectual Property Administration (CNIPA) on Dec. 14, 2020, the disclosures of which are incorporated herein by reference in their entireties.

### TECHNICAL FIELD

The present application belongs to the field of biomedical technology and relates to an anti-B7H3 chimeric antigen receptor and a use thereof.

### BACKGROUND

B7H3 is a type I transmembrane protein, which belongs to a B7 immune co-stimulation and co-suppression family. B7H3 has an immunosuppressive function, which can reduce type I interferon (IFN) released from T cells and reduce cytotoxicity of NK cells. B7H3 proteins have limited expression in normal tissues (such as prostate, breast, placenta, liver, colon and lymphoid organs) but are abnormally and highly expressed in most malignant tumors. B7H3 expression can be detected in non-small-cell lung cancer cell lines and tumor tissues. In the tumor tissues expressing B7H3, the number of infiltrating lymphoid cells is significantly reduced and positively correlated with lymph node metastasis (Sun Y, Wang Y, Zhao J, et al. B7-H3 and B7-H4 expression in non-small-cell lung cancer[J]. Lung Cancer, 2006, 53(2): 143-151; Zhao Wenjian, Chen Chunyan, Sui Wenyan, et al. Advances in B7-H3 and its relationship with tumors [J]. Medical Recapitulate, 2009, 15(22): 3430-3433.).

Generally, high expression of B7H3 in tumor cells is closely associated with reduced tumor-infiltrating lymphocytes, accelerated cancer progression and clinical outcomes of malignant tumors (nervous system tumor, melanoma, lung cancer, head and neck cancer, colorectal cancer, pancreatic cancer, prostate cancer, ovarian cancer, lung cancer and clear cell renal cell carcinoma). B7H3 has become a potential target for cancer immunotherapy due to widespread expression in a variety of tumors. However, there are few reports on B7H3-targeting immunotherapy at present.

### SUMMARY

The present application provides an anti-B7H3 chimeric antigen receptor and a use thereof. The anti-B7H3 chimeric antigen receptor uses an anti-B7H3 antibody having a binding ability to human B7H3 as an antigen-binding domain, which can bind not only free B7H3 proteins but also B7H3 proteins on a cell surface and has an important application prospect in the field of tumor treatment.

In a first aspect, the present application provides an anti-B7H3 chimeric antigen receptor. The anti-B7H3 chimeric antigen receptor includes an antigen-binding domain, a hinge region, a transmembrane domain and a signaling domain;

wherein the antigen-binding domain is an anti-B7H3 antibody.

In the present application, the anti-B7H3 antibody having a binding ability to B7H3 is used as the antigen-binding domain of the chimeric antigen receptor so that the chimeric antigen receptor can specifically bind to B7H3-positive tumor cells, thereby achieving a specific targeting effect on B7H3-positive tumors.

In some specific embodiments, the antigen-binding domain includes amino acid sequences shown in SEQ ID NO: 1 and SEQ ID NO: 2, wherein SEQ ID NO: 1 and SEQ ID NO: 2 are linked by a linker peptide to form an anti-B7H3 antibody H26B6; wherein

SEQ ID NO: 1:
EIVLTQSPATLSLSPGERATLSCSASSSVSYMQWYQQKPGLAPRLLIYDT
SKLTSGIPDRFSGSGSGTDFTLTISRLEPEDFAVYYCQQWSSNPLTFGGG
TKVEIKRTV;
SEQ ID NO: 2:
EVQLVQSGAEVKKPGASVKVSCKASGYTFTEYTMHWVRQAPGQGLEWIGG
INPNTGGTTYNQKFNGRVTMTRDTSISTAYMELSSLRSEDTAVYYCTRPY
RDDGGFHWYFDVWGQGTLVTVSS.

In some specific embodiments, the antigen-binding domain includes amino acid sequences shown in SEQ ID NO: 3 and SEQ ID NO: 4, wherein SEQ ID NO: 3 and SEQ ID NO: 4 are linked by a linker peptide to form an anti-B7H3 antibody H2B8; wherein

SEQ ID NO: 3:
DIQLTQSPSFLSASVGDRVTINCRASKTISNYLAWYQQKPGKAPKLLIYS
GSTLQSGVPSRFSGSGSGTDFTLTISSLQPEDFATYYCQQHHEYPLTFGG
GTKVEIKRTV;
SEQ ID NO: 4:
QVQLVQSGAEVKKPGASVKVSCKVSGYTFTDGAMHWVRQAPGQGLEWIGI
INTNSGNTNYNQKFQGRVTMTRDTSISTAYMELSRLRSEDTAVYYCARGV
FYYGYGAWFAYWGQGTLVTVSS.

In some specific embodiments, the antigen-binding domain includes amino acid sequences shown in SEQ ID NO: 5 and SEQ ID NO: 6, wherein SEQ ID NO: 5 and SEQ ID NO: 6 are linked by a linker peptide to form an anti-B7H3 antibody 26H6; wherein

SEQ ID NO: 5:
QIVLTQSPAVMSTSPGEKVTMTCSASSSVSYMQWYQQKSGTSPKRWIYDT
SKLTSGVPARFSGSGSGTSYSLTISSMEAEDAATYYCQQWSSNPLTFGAG
TKLELK;
SEQ ID NO: 6:
EVQLQQSGPELVKPGASVKISCKTSGYAFTEYTMHWVKQSQGKSLEWIGG
INPNTGGTTYNQKFNGKATLTVDRSSSTAYMELRSLTSEDSAVYYCTRPY
RDDGGFHWYFDVWGAGTAVTVSS.

In some specific embodiments, the antigen-binding domain includes amino acid sequences shown in SEQ ID NO: 7 and SEQ ID NO: 8, wherein SEQ ID NO: 7 and SEQ ID NO: 8 are linked by a linker peptide to form an anti-B7H3 antibody 2B8; wherein

SEQ ID NO: 7:
DVQITQSPSYLTASPGETIIINCRASKTISNYLAWYQEKPGKTNKLLIYS
GSTLQSGIPSRFSGSGSDTDFTLTISSLEPEDFAMYYCQQHHEYPLTFGA
GTKLELK;
SEQ ID NO: 8:
QVQLQQSGPELVRPGVSVKISCKVSGYTFTDGAMHWVKRSHAKSLEWIGI
INTNSGNTNYNQKFQGKATMTVDKSSSTAYMELARLTSEDSAIYYCARGV
FYYGYGAWFAYWGQGTLVTVSA.

In some specific embodiments, the antigen-binding domain includes amino acid sequences shown in SEQ ID NO: 9 and SEQ ID NO: 10, wherein SEQ ID NO: 9 and SEQ ID NO: 10 are linked by a linker peptide to form an anti-B7H3 antibody 23H1; wherein

SEQ ID NO: 9:
DIVMTQSPSSLTVTAGENVTMSCKSSQTLLNNGNQKNFLTWYQQKPGQPP
KLLIYLASTRESGVPDRFTGSGSGTDFTLTISSVQAEDLAVYYCONDYTY
PLTFGAGTKLELK;
SEQ ID NO: 10:
EVKLVESGGGLVQPGGSLRLSCATSGFTFTDYYMSWVRQPPGKALEWLGF
IRNKVNDYTTEYSVSVKGRFTISRDNSQTILYLQMNTLRAEDSATYYCAR
DSPYRPFAYWGQGTLVTVSA.

In some specific embodiments, the antigen-binding domain includes amino acid sequences shown in SEQ ID NO: 11 and SEQ ID NO: 12, wherein SEQ ID NO: 11 and SEQ ID NO: 12 are linked by a linker peptide to form an anti-B7H3 antibody 6F7; wherein

SEQ ID NO: 11:
DIQMTQSPSSLTVTAGEKVTMSCKSSQSLLNSGNQNNYLTWYQQKPGQPP
KLLIYLASTRDSGVPDRFTGSGSGTDFTLTISSVQAEDLAVYYCQNDYTY
PLTFGAGTKLELK;
SEQ ID NO: 12:
EVKLVESGGGLVQPGGSLRLSCATSGFTFTDYYMSWVRQPPGKALEWLGF
IRNKANGYTTEYSASVKGRFTISSDDSQSILYLQMNTLRAEDSATYYCAR
DSHYRPFAYWGQGTLVTVSA.

In some specific embodiments, the antigen-binding domain includes amino acid sequences shown in SEQ ID NO: 13 and SEQ ID NO: 14, wherein SEQ ID NO: 13 and SEQ ID NO: 14 are linked by a linker peptide to form an anti-B7H3 antibody Enoblituzumab (Eno); wherein

SEQ ID NO: 13:
DIQLTQSPSFLSASVGDRVTITCKASQNVDTNVAWYQQKPGKAPKALIYS
ASYRYSGVPSRFSGSGSGTDFTLTISSLQPEDFATYYCQQYNNYPFTFGQ
GTKLEIK;
SEQ ID NO: 14:
EVQLVESGGGLVQPGGSLRLSCAASGFTFSSFGMHWVRQAPGKGLEWVAY
ISSDSSAIYYADTVKGRFTISRDNAKNSLYLQMNSLRDEDTAVYYCGRGR
ENIYYGSRLDYWGQGTTVTVSS.

In some specific embodiments, the antigen-binding domain includes amino acid sequences shown in SEQ ID NO: 15 and SEQ ID NO: 16, wherein SEQ ID NO: 15 and SEQ ID NO: 16 are linked by a linker peptide to form an anti-B7H3 antibody huM30; wherein

SEQ ID NO: 15:
EIVLTQSPATLSLSPGERATLSCRASSRLIYMHWYQQKPGQAPRPLIYAT
SNLASGIPARFSGSGSGTDFTLTISSLEPEDFAVYYCQQWNSNPPTFGQG
TKVEIK;
SEQ ID NO: 16:
QVQLVQSGAEVKKPGSSVKVSCKASGYTFTNYVMHWVRQAPGQGLEWMGY
INPYNDDVKYNEKFKGRVTITADESTSTAYMELSSLRSEDTAVYYCARWG
YYGSPLYYFDYWGQGTLVTVSS.

Preferably, the hinge region includes a CD8α hinge region.

Preferably, the transmembrane domain includes a CD8α transmembrane region and/or a CD28 transmembrane region.

Preferably, the signaling domain includes CD3ζ.

Preferably, the signaling domain further includes any one or a combination of at least two of 4-1BB, a CD28 intracellular region, DAP10 or OX40.

Preferably, the anti-B7H3 chimeric antigen receptor further includes a signal peptide.

Preferably, the signal peptide includes any one of an IgGκ light chain signal peptide, a CD8α signal peptide, a GM-CSF signal peptide, an HSA signal peptide, an IgG heavy chain signal peptide, an IgG light chain signal peptide, a CD33 signal peptide, an IL-2 signal peptide or an insulin signal peptide.

As a preferred technical solution, the present application provides the anti-B7H3 chimeric antigen receptor. The anti-B7H3 chimeric antigen receptor includes the signal peptide, the anti-B7H3 antibody, the CD8α hinge region, the CD8α transmembrane region, 4-1BB and CD3ζ.

In some specific embodiments, the anti-B7H3 chimeric antigen receptor is H26B6-CAR, which is formed of the IgGκ light chain signal peptide, the anti-B7H3 antibody H26B6, the CD8α hinge region, the CD8α transmembrane region, 4-1BB and CD3ζ in tandem. H26B6-CAR includes an amino acid sequence shown in SEQ ID NO: 17; wherein

SEQ ID NO: 17:
MDMRVPAQLLGLLLLWLRGARCEIVLTQSPATLSLSPGERATLSCSASSS
VSYMQWYQQKPGLAPRLLIYDTSKLTSGIPDRFSGSGSGTDFTLTISRLE
PEDFAVYYCQQWSSNPLTFGGGTKVEIKRTVGGGGSGGGGSGGGGSEVQL
VQSGAEVKKPGASVKVSCKASGYTFTEYTMHWVRQAPGQGLEWIGGINPN
TGGTTYNQKFNGRVTMTRDTSISTAYMELSSLRSEDTAVYYCTRPYRDDG
GFHWYFDVWGQGTLVTVSSTTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR.

In some specific embodiments, the anti-B7H3 chimeric antigen receptor is H2B8-CAR, which is formed of the IgGκ light chain signal peptide, the anti-B7H3 antibody H2B8, the CD8α hinge region, the CD8α transmembrane region, 4-1BB and CD3ζ in tandem. H2B8-CAR includes an amino acid sequence shown in SEQ ID NO: 18; wherein

SEQ ID NO: 18:
MDMRVPAQLLGLLLLWLRGARCDIQLTQSPSFLSASVGDRVTINCRASKT
ISNYLAWYQQKPGKAPKLLIYSGSTLQSGVPSRFSGSGSGTDFTLTISSL
QPEDFATYYCQQHHEYPLTFGGGTKVEIKRTVGGGGSGGGGSGGGGSQVQ
LVQSGAEVKKPGASVKVSCKVSGYTFTDGAMHWVRQAPGQGLEWIGIINT
NSGNTNYNQKFQGRVTMTRDTSISTAYMELSRLRSEDTAVYYCARGVFYY
GYGAWFAYWGQGTLVTVSSTTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR.

In some specific embodiments, the anti-B7H3 chimeric antigen receptor is L26B6-CAR, which is formed of a HuIgGκ light chain signal peptide, the anti-B7H3 antibody 26B6, the CD8α hinge region, the CD8α transmembrane region, 4-1BB and CD3ζ in tandem. L26B6-CAR includes an amino acid sequence shown in SEQ ID NO: 19; wherein

SEQ ID NO: 19:
METPAQLLFLLLLWLPDTTGQIVLTQSPAVMSTSPGEKVTMTCSASSSVS
YMQWYQQKSGTSPKRWIYDTSKLTSGVPARFSGSGSGTSYSLTISSMEAE
DAATYYCQQWSSNPLTFGAGTKLELKGGGGSGGGGSGGGGSEVQLQQSGP
ELVKPGASVKISCKTSGYAFTEYTMHWVKQSQGKSLEWIGGINPNTGGTT
YNQKFNGKATLTVDRSSSTAYMELRSLTSEDSAVYYCTRPYRDDGGFHWY
FDVWGAGTAVTVSSTTTPAPRPPTPAPTIASQPLSLRPEACRPAAGGAVH
TRGLDFACDIYIWAPLAGTCGVLLLSLVITLYCKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR.

In some specific embodiments, the anti-B7H3 chimeric antigen receptor is 26B6-CAR, which is formed of the CD8α signal peptide, the anti-B7H3 antibody 26B6, the CD8α hinge region, the CD8α transmembrane region, 4-1BB and CD3ζ in tandem. 26B6-CAR includes an amino acid sequence shown in SEQ ID NO: 20; wherein

SEQ ID NO: 20:
MALPVTALLLPLALLLHAARPQIVLTQSPAVMSTSPGEKVTMTCSASSSV
SYMQWYQQKSGTSPKRWIYDTSKLTSGVPARFSGSGSGTSYSLTISSMEA
EDAATYYCQQWSSNPLTFGAGTKLELKGGGGSGGGGSGGGGSEVQLQQSG
PELVKPGASVKISCKTSGYAFTEYTMHWVKQSQGKSLEWIGGINPNTGGT
TYNQKFNGKATLTVDRSSSTAYMELRSLTSEDSAVYYCTRPYRDDGGFHW
YFDVWGAGTAVTVSSTTTPAPRPPTPAPTIASQPLSLRPEACRPAAGGAV
HTRGLDFACDIYIWAPLAGTCGVLLLSLVITLYCKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR.

In some specific embodiments, the anti-B7H3 chimeric antigen receptor is L2B8-CAR, which is formed of the IgGκ light chain signal peptide, the anti-B7H3 antibody 2B8, the CD8α hinge region, the CD8α transmembrane region, 4-1BB and CD3ζ in tandem. L2B8-CAR includes an amino acid sequence shown in SEQ ID NO: 21; wherein

SEQ ID NO: 21:
MDMRVPAQLLGLLLLWLRGARCDVQITQSPSYLTASPGETIIINCRASKT
ISNYLAWYQEKPGKTNKLLIYSGSTLQSGIPSRFSGSGSDTDFTLTISSL
EPEDFAMYYCQQHHEYPLTFGAGTKLELKGGGGSGGGGSGGGGSQVQLQQ
SGPELVRPGVSVKISCKVSGYTFTDGAMHWVKRSHAKSLEWIGIINTNSG
NTNYNQKFQGKATMTVDKSSSTAYMELARLTSEDSAIYYCARGVFYYGYG
AWFAYWGQGTLVTVSATTTPAPRPPTPAPTIASQPLSLRPEACRPAAGGA
VHTRGLDFACDIYIWAPLAGTCGVLLLSLVITLYCKRGRKKLLYIFKQPF
MRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR.

In some specific embodiments, the anti-B7H3 chimeric antigen receptor is 2B8-CAR, which is formed of the CD8α signal peptide, the anti-B7H3 antibody 2B8, the CD8α hinge region, the CD8α transmembrane region, 4-1BB and CD3ζ in tandem. 2B8-CAR includes an amino acid sequence shown in SEQ ID NO: 22; wherein

SEQ ID NO: 22:
MALPVTALLLPLALLLHAARPDVQITQSPSYLTASPGETIIINCRASKTI
SNYLAWYQEKPGKTNKLLIYSGSTLQSGIPSRFSGSGSDTDFTLTISSLE
PEDFAMYYCQQHHEYPLTFGAGTKLELKGGGGSGGGGSGGGGSQVQLQQS
GPELVRPGVSVKISCKVSGYTFTDGAMHWVKRSHAKSLEWIGIINTNSGN
TNYNQKFQGKATMTVDKSSSTAYMELARLTSEDSAIYYCARGVFYYGYGA
WFAYWGQGTLVTVSATTTPAPRPPTPAPTIASQPLSLRPEACRPAAGGAV
HTRGLDFACDIYIWAPLAGTCGVLLLSLVITLYCKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR.

In some specific embodiments, the anti-B7H3 chimeric antigen receptor is L23H1-CAR, which is formed of an IgGκ signal peptide, the anti-B7H3 antibody 23H1, the CD8α hinge region, the CD8α transmembrane region, 4-1BB and CD3ζ in tandem. L23H1-CAR includes an amino acid sequence shown in SEQ ID NO: 23; wherein

SEQ ID NO: 23:
MDMRVPAQLLGLLLLWLRGARCDIVMTQSPSSLTVTAGENVTMSCKSSQT
LLNNGNQKNFLTWYQQKPGQPPKLLIYLASTRESGVPDRFTGSGSGTDFT
LTISSVQAEDLAVYYCQNDYTYPLTFGAGTKLELKGGGGSGGGGSGGGGS
EVKLVESGGGLVQPGGSLRLSCATSGFTFTDYYMSWVRQPPGKALEWLGF
IRNKVNDYTTEYSVSVKGRFTISRDNSQTILYLQMNTLRAEDSATYYCAR
DSPYRPFAYWGQGTLVTVSATTTPAPRPPTPAPTIASQPLSLRPEACRPA
AGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAE
AYSEIGMKGERRRGKGHDGLYQGLSTATKDTYDALHMQALPPR.

In some specific embodiments, the anti-B7H3 chimeric antigen receptor is 23H1-CAR, which is formed of the CD8α signal peptide, the anti-B7H3 antibody 23H1, the CD8α hinge region, the CD8α transmembrane region, 4-1BB and CD3ζ in tandem. 23H1-CAR includes an amino acid sequence shown in SEQ ID NO: 24; wherein

SEQ ID NO: 24:
MALPVTALLLPLALLLHAARPDIVMTQSPSSLTVTAGENVTMSCKSSQTL
LNNGNQKNFLTWYQQKPGQPPKLLIYLASTRESGVPDRFTGSGSGTDFTL
TISSVQAEDLAVYYCQNDYTYPLTFGAGTKLELKGGGGSGGGGSGGGGSE
VKLVESGGGLVQPGGSLRLSCATSGFTFTDYYMSWVRQPPGKALEWLGFI
RNKVNDYTTEYSVSVKGRFTISRDNSQTILYLQMNTLRAEDSATYYCARD
SPYRPFAYWGQGTLVTVSATTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR.

In some specific embodiments, the anti-B7H3 chimeric antigen receptor is L6F7-CAR, which is formed of the IgGκ signal peptide, the anti-B7H3 antibody 6F7, the CD8α hinge region, the CD8α transmembrane region, 4-1BB and CD3ζ in tandem. L6F7-CAR includes an amino acid sequence shown in SEQ ID NO: 25; wherein

SEQ ID NO: 25:
MDMRVPAQLLGLLLLWLRGARCDIQMTQSPSSLTVTAGEKVTMSCKSSQS
LLNSGNQNNYLTWYQQKPGQPPKLLIYLASTRDSGVPDRFTGSGSGTDFT
LTISSVQAEDLAVYYCQNDYTYPLTFGAGTKLELKGGGGSGGGGSGGGGS
EVKLVESGGGLVQPGGSLRLSCATSGFTFTDYYMSWVRQPPGKALEWLGF
IRNKANGYTTEYSASVKGRFTISSDDSQSILYLQMNTLRAEDSATYYCAR
DSHYRPFAYWGQGTLVTVSATTTPAPRPPTPAPTIASQPLSLRPEACRPA
AGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAE
AYSEIGMKGERRRGKGHDGLYQGLSTATKDTYDALHMQALPPR.

In some specific embodiments, the anti-B7H3 chimeric antigen receptor is 6F7-CAR, which is formed of the CD8α signal peptide, the anti-B7H3 antibody 6F7, the CD8α hinge region, the CD8α transmembrane region, 4-1BB and CD3ζ in tandem. 6F7-CAR includes an amino acid sequence shown in SEQ ID NO: 26; wherein

SEQ ID NO: 26:
MALPVTALLLPLALLLHAARPDIQMTQSPSSLTVTAGEKVTMSCKSSQSL
LNSGNQNNYLTWYQQKPGQPPKLLIYLASTRDSGVPDRFTGSGSGTDFTL
TISSVQAEDLAVYYCQNDYTYPLTFGAGTKLELKGGGGSGGGGSGGGGSE
VKLVESGGGLVQPGGSLRLSCATSGFTFTDYYMSWVRQPPGKALEWLGFI
RNKANGYTTEYSASVKGRFTISSDDSQSILYLQMNTLRAEDSATYYCARD
SHYRPFAYWGQGTLVTVSATTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR.

In some specific embodiments, the anti-B7H3 chimeric antigen receptor is Eno-CAR, which is formed of the CD8α signal peptide, the anti-B7H3 antibody Enoblituzumab, the CD8α hinge region, the CD8α transmembrane region, 4-1BB and CD3ζ in tandem. Eno-CAR includes an amino acid sequence shown in SEQ ID NO: 27; wherein

SEQ ID NO: 27:
MALPVTALLLPLALLLHAARPDIQLTQSPSFLSASVGDRVTITCKASQNV
DTNVAWYQQKPGKAPKALIYSASYRYSGVPSRFSGSGSGTDFTLTISSLQ
PEDFATYYCQQYNNYPFTFGQGTKLEIKGGGGSGGGGSGGGGSEVQLVES
GGGLVQPGGSLRLSCAASGFTFSSFGMHWVRQAPGKGLEWVAYISSDSSA
IYYADTVKGRFTISRDNAKNSLYLQMNSLRDEDTAVYYCGRGRENIYYGS
RLDYWGQGTTVTVSSTTTPAPRPPTPAPTIASQPLSLRPEACRPAAGGAV
HTRGLDFACDIYIWAPLAGTCGVLLLSLVITLYCKRGRKKLLYIFKQPFM
RPVQTTQEEDGCSCRFPEEEEGGCELRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR.

In some specific embodiments, the anti-B7H3 chimeric antigen receptor is huM30-CAR, which is formed of the CD8α signal peptide, the anti-B7H3 antibody huM30, the CD8α hinge region, the CD8α transmembrane region, 4-1BB and CD3ζ in tandem. huM30-CAR includes an amino acid sequence shown in SEQ ID NO: 28; wherein

SEQ ID NO: 28:
MALPVTALLLPLALLLHAARPEIVLTQSPATLSLSPGERATLSCRASSRL
IYMHWYQQKPGQAPRPLIYATSNLASGIPARFSGSGSGTDFTLTISSLEP
EDFAVYYCQQWNSNPPTFGQGTKVEIKGGGGSGGGGSGGGGSQVQLVQSG
AEVKKPGSSVKVSCKASGYTFTNYVMHWVRQAPGQGLEWMGYINPYNDDV
KYNEKFKGRVTITADESTSTAYMELSSLRSEDTAVYYCARWGYYGSPLYY
FDYWGQGTLVTVSSTTTPAPRPPTPAPTIASQPLSLRPEACRPAAGGAVH
TRGLDFACDIYIWAPLAGTCGVLLLSLVITLYCKRGRKKLLYIFKQPFMR
PVQTTQEEDGCSCRFPEEEEGGCELRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR.

In a second aspect, the present application provides a nucleic acid molecule. The nucleic acid molecule includes a coding gene of the anti-B7H3 chimeric antigen receptor according to the first aspect.

In some specific embodiments, the nucleic acid molecule includes a nucleic acid sequence shown in SEQ ID NO: 29, which is a coding gene of H26B8-CAR.

In some specific embodiments, the nucleic acid molecule includes a nucleic acid sequence shown in SEQ ID NO: 30, which is a coding gene of H2B8-CAR.

In some specific embodiments, the nucleic acid molecule includes a nucleic acid sequence shown in SEQ ID NO: 31, which is a coding gene of L26B6-CAR.

In some specific embodiments, the nucleic acid molecule includes a nucleic acid sequence shown in SEQ ID NO: 32, which is a coding gene of 26B6-CAR.

In some specific embodiments, the nucleic acid molecule includes a nucleic acid sequence shown in SEQ ID NO: 33, which is a coding gene of L2B8-CAR.

In some specific embodiments, the nucleic acid molecule includes a nucleic acid sequence shown in SEQ ID NO: 34, which is a coding gene of 2B8-CAR.

In some specific embodiments, the nucleic acid molecule includes a nucleic acid sequence shown in SEQ ID NO: 35, which is a coding gene of L23H1-CAR.

In some specific embodiments, the nucleic acid molecule includes a nucleic acid sequence shown in SEQ ID NO: 36, which is a coding gene of 23H1-CAR.

In some specific embodiments, the nucleic acid molecule includes a nucleic acid sequence shown in SEQ ID NO: 37, which is a coding gene of L6F7-CAR.

In some specific embodiments, the nucleic acid molecule includes a nucleic acid sequence shown in SEQ ID NO: 38, which is a coding gene of 6F7-CAR.

In some specific embodiments, the nucleic acid molecule includes a nucleic acid sequence shown in SEQ ID NO: 39, which is a coding gene of Eno-CAR.

In some specific embodiments, the nucleic acid molecule includes a nucleic acid sequence shown in SEQ ID NO: 40, which is a coding gene of huM30-CAR.

In a third aspect, the present application provides an expression vector. The expression vector includes the nucleic acid molecule according to the second aspect.

Preferably, the expression vector is any one of a lentiviral vector, a retroviral vector or an adeno-associated viral vector containing the nucleic acid molecule according to the second aspect, preferably the lentiviral vector.

In a fourth aspect, the present application provides a recombinant lentivirus. The recombinant lentivirus is prepared from a mammalian cell transfected with the expression vector according to the third aspect and a helper plasmid.

In a fifth aspect, the present application provides a chimeric antigen receptor T cell. The chimeric antigen receptor T cell expresses the anti-B7H3 chimeric antigen receptor according to the first aspect.

In the present application, the T cell expressing the anti-B7H3 chimeric antigen receptor targets B7H3-positive tumor cells using the antigen-binding domain of the chimeric antigen receptor and exerts a killing function of the T cell, thereby achieving a killing effect on B7H3-positive tumors.

Preferably, a genome of the chimeric antigen receptor T cell is integrated with the nucleic acid molecule according to the second aspect.

Preferably, the chimeric antigen receptor T cell includes the expression vector according to the third aspect and/or the recombinant lentivirus according to the fourth aspect.

In a sixth aspect, the present application provides a pharmaceutical composition. The pharmaceutical composition includes the chimeric antigen receptor T cell according to the fifth aspect.

Preferably, the pharmaceutical composition further includes any one or a combination of at least two of a pharmaceutically acceptable carrier, excipient or diluent.

In a seventh aspect, the present application provides a use of the anti-B7H3 chimeric antigen receptor according to the first aspect, the nucleic acid molecule according to the second aspect, the expression vector according to the third aspect, the recombinant lentivirus according to the fourth aspect, the chimeric antigen receptor T cell according to the fifth aspect or the pharmaceutical composition according to the sixth aspect for preparing a malignant tumor treatment drug.

Preferably, the malignant tumor includes any one or a combination of at least two of acute lymphoblastic leukemia, myeloid leukemia, melanoma, neuroblastoma, non-small-cell lung cancer, nasopharyngeal carcinoma, breast cancer, colorectal cancer, liver cancer, pancreatic cancer or cervical cancer.

Compared with the prior art, the present application has the beneficial effects described below.
(1) In the present application, an anti-human B7H3 antibody is used as the antigen-binding domain to construct a CAR molecule, and the T cell expressing the anti-B7H3 CAR has a significant killing effect on the B7H3-positive tumor cells at different effector to target ratios, wherein H26B6-CAR-T has an optimal killing function.
(2) The anti-B7H3 CAR-T cell of the present application secretes a large number of cytokines IFN-γ after co-culture with the tumor cells, which indirectly proves the killing effect of CAR-T on the tumor cells.
(3) Anti-B7H3 CAR-T, especially H26B6-CAR-T, of the present application has a significant in vivo pharmacodynamic effect. After an administration to tumor model mice, H26B6-CAR-T can significantly inhibit the growth of the tumor cells, promote the apoptosis of the tumor cells and secrete the cytokines IFN-γ, and effectively eliminating the tumor cells.

### BRIEF DESCRIPTION OF DRAWINGS

FIG. 1 is a structure diagram of an anti-B7H3-CAR molecule.

FIG. 2 is a map of a recombinant lentiviral vector pCDH-EF1-anti-B7H3-CAR.

FIG. 3A is a flow cytometry diagram of H26B6-CAR-T, H2B8-CAR-T, L2B8-CAR-T and L26B6-CAR-T, FIG. 3B is a flow cytometry diagram of Eno-CAR-T and huM30-CAR-T, FIG. 3C is a flow cytometry diagram of huM30-CAR-T and 26B6-CAR-T in another experiment, and FIG. 3D is a flow cytometry diagram of huM30-CAR-T, L2B8-CAR-T and 2B8-CAR-T in another experiment.

FIG. 4A illustrates killing efficiency of H26B6-CAR-T, H2B8-CAR-T, L2B8-CAR-T and L26B6-CAR-T on human liver cancer cells HepG2 at different effector to target ratios, FIG. 4B illustrates killing efficiency of H26B6-CAR-T, H2B8-CAR-T, L2B8-CAR-T and L26B6-CAR-T on human pancreatic cancer cells PL45 at different effector to target ratios, FIG. 4C illustrates killing efficiency of H26B6-CAR-T, H2B8-CAR-T, L2B8-CAR-T and L26B6-CAR-T on human cervical cancer cells SiHa at different effector to target ratios, FIG. 4D illustrates killing efficiency of huM30-CAR-T, 2B8-CAR-T and T cells on target cells at different effector to target ratios, FIG. 4E illustrates killing efficiency of huM30-CAR-T, 2B8-CAR-T and T cells on target cells at different effector to target ratios, FIG. 4F illustrates killing efficiency of huM30-CAR-T, 26B6-CAR-T and T cells on PL45 at different effector to target ratios, FIG. 4G illustrates killing efficiency of huM30-CAR-T, 26B6-CAR-T and T cells on PC9 at different effector to target ratios, FIG. 4H illustrates killing efficiency of huM30-CAR-T, 26B6-CAR-T and T cells on SiHa at different effector to target ratios, FIG. 41 illustrates killing efficiency of huM30-CAR-T, 26B6-CAR-T and T cells on HepG2.0 at different effector to target ratios, FIG. 4J illustrates killing efficiency of huM30-CAR-T, 2B8-CAR-T, L2B8-CAR-T and T cells on PL45 at different effector to target ratios, FIG. 4K illustrates killing efficiency of huM30-CAR-T, 2B8-CAR-T, L2B8-CAR-T and T cells on PC9 at different effector to target ratios, FIG. 4L illustrates killing efficiency of huM30-CAR-T, 2B8-CAR-T, L2B8-CAR-T and T cells on SiHa at different effector to target ratios, and FIG. 4M illustrates killing efficiency of huM30-CAR-T, 2B8-CAR-T, L2B8-CAR-T and T cells on HepG2 at different effector to target ratios.

FIG. 5A illustrates the secretion of IFN-γ after Eno-CAR-T is co-incubated with target cells according to different effector to target ratios, and FIG. 5B illustrates the secretion of IFN-γ after huM30-CAR-T is co-incubated with target cells according to different effector to target ratios.

FIG. 6A illustrates variations of weights of mice during an administration of A375 tumor models, FIG. 6B illustrates variations of weights of mice during an administration of Hep3B tumor models, and FIG. 6C illustrates variations of weights of mice during an administration of SiHa tumor models.

FIG. 7A illustrates variations of tumor volumes during an administration of A375 tumor models, FIG. 7B illustrates variations of tumor volumes during an administration of Hep3B tumor models, and FIG. 7C illustrates variations of tumor volumes during an administration of SiHa tumor models.

FIG. 8A illustrates intravital imaging fluorescence data of mice during an administration of A375 tumor models, FIG. 8B illustrates intravital imaging fluorescence data of mice during an administration of Hep3B tumor models, and FIG. 8C illustrates intravital imaging fluorescence data of mice during an administration of SiHa tumor models.

FIG. 9A illustrates a secretion level of IFN-γ in serum of mice during an administration of A375 tumor models, FIG. 9B illustrates a secretion level of IFN-γ in serum of mice during an administration of Hep3B tumor models, and FIG. 9C illustrates a secretion level of IFN-γ in serum of mice during an administration of SiHa tumor models.

### DETAILED DESCRIPTION

To further elaborate on the technical means adopted and effects achieved in the present application, the present application is further described below in conjunction with examples and drawings. It is to be understood that the specific examples set forth below are intended to explain the present application and not to limit the present application.

Experiments without specific techniques or conditions specified in the examples are conducted according to techniques or conditions described in the literature in the art or a product specification. The reagents or instruments used herein without manufacturers specified are conventional products commercially available from proper channels.

### Example 1 Preparation of CAR-T Cells

In this example, anti-B7H3 antibodies H26B6, H2B8, 26B6, 2B8, 23H1, 6F7, Enoblituzumab (Eno) and huM30 were selected as antigen-binding domains to construct CAR molecules. 26B6 and humanized H26B6, 2B8 and humanized H2B8, 23H1 and 6F7, which have a significant binding ability to B7H3, can bind not only free B7H3 proteins but also B7H3 proteins on a cell surface. huM30 is a humanized B7H3 antibody (CN103687945B) of Daiichi Sankyo Co., Ltd. in Japan, and a phase I clinical trial is being conducted for the treatment of B7H3-positive solid tumors (NCT02192567). Enoblituzumab (MGA271), a brand-new monoclonal antibody optimized by an immune molecule and aimed at a B7H3 target, is developed by MacroGenics using an exclusive Fc optimization technology and has a unique antibody advantage and a therapeutic potential. With no such drug having been approved in the world, Enoblituzumab represents a leading B7H3 antibody drug in the world.

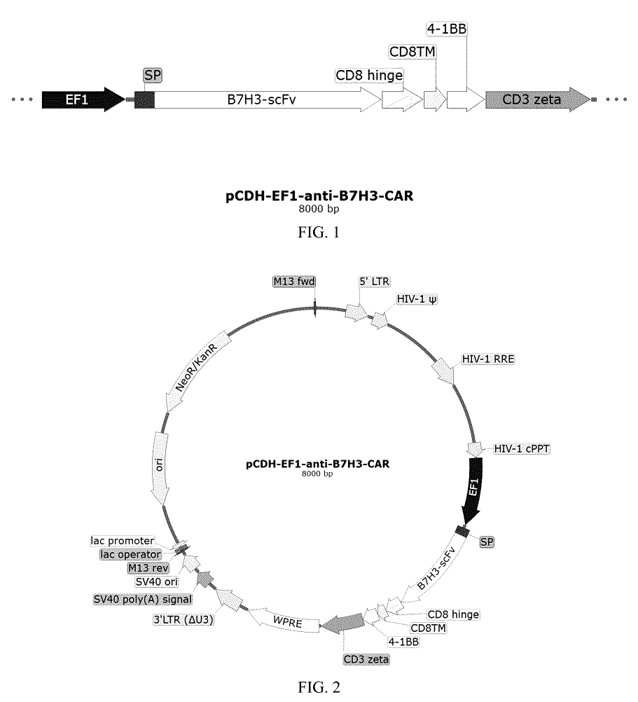

In this example, the above anti-B7H3 antibody was used as the antigen-binding domain of the CAR molecule and combined with a hinge region, a transmembrane domain and a signaling domain to construct the anti-B7H3 CAR molecule shown in FIG. 1.

Specifically, the CAR molecule is:
a IgGκ light chain signal peptide, H26B6, CD8α hinge region, CD8α transmembrane region, 4-1BB and CD3ζ (SEQ ID NO: 17);
b IgGκ light chain signal peptide, H2B8, CD8α hinge region, CD8α transmembrane region, 4-1BB and CD3ζ (SEQ ID NO: 18);
c HuIgGκ light chain signal peptide, L26B6, CD8α hinge region, CD8α transmembrane region, 4-1BB and CD3ζ (SEQ ID NO: 19);
d CD8α signal peptide, 26B6, CD8α hinge region, CD8α transmembrane region, 4-1BB and CD3ζ (SEQ ID NO: 20);
e IgGκ light chain signal peptide, L2B8, CD8α hinge region, CD8α transmembrane region, 4-1BB and CD3ζ (SEQ ID NO: 21);
f CD8α signal peptide, 2B8, CD8α hinge region, CD8α transmembrane region, 4-1BB and CD3ζ (SEQ ID NO: 22);
g IgGκ light chain signal peptide, L23H1, CD8α hinge region, CD8α transmembrane region, 4-1BB and CD3ζ (SEQ ID NO: 23);
h CD8α signal peptide, 23H1, CD8α hinge region, CD8α transmembrane region, 4-1BB and CD3ζ (SEQ ID NO: 24);
i IgGκ light chain signal peptide, L6F7, CD8α hinge region, CD8α transmembrane region, 4-1BB and CD3ζ (SEQ ID NO: 25);
j CD8α signal peptide, 6F7, CD8α hinge region, CD8α transmembrane region, 4-1BB and CD3ζ (SEQ ID NO: 26);
k CD8α signal peptide, Eno, CD8α hinge region, CD8α transmembrane region, 4-1BB and CD3ζ (SEQ ID NO: 27);
l CD8α signal peptide, huM30, CD8α hinge region, CD8α transmembrane region, 4-1BB and CD3ζ (SEQ ID NO: 28).

Coding genes of the above CAR molecules were synthesized through a gene synthesis, and the synthesized coding genes of the CAR molecules were cloned into a lentiviral vector pCDH through steps such as PCR, enzyme digestion and recombination to obtain the recombinant lentiviral vector pCDH-EF1-anti-B7H3-CAR as shown in FIG. 2.

The recombinant lentiviral plasmid vector was packaged into recombinant lentiviral particles using 293T cells and helper plasmids, and the activated T cells were infected to obtain CAR-T cells H26B6-CAR-T, H2B8-CAR-T, L26B6-CAR-T, 26B6-CAR-T, L2B8-CAR-T, 2B8-CAR-T, L23H1-CAR-T, 23H1-CAR-T, L6F7-CAR-T, 6F7-CAR-T, Eno-CAR-T and huM30-CAR-T expressing different CARs.

### Example 2 Expression Efficiency of CAR in CAR-T Cells

Expression rates of CAR in CAR-T cells were detected using a flow cytometer.

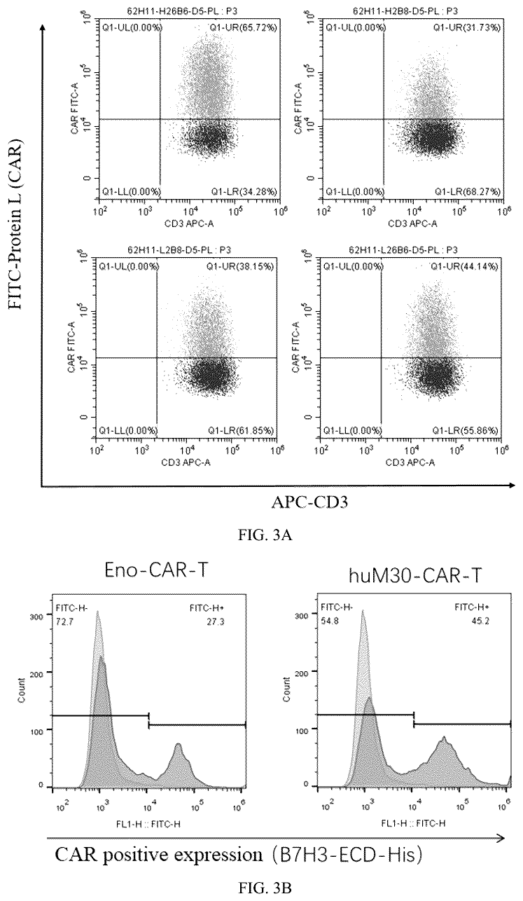

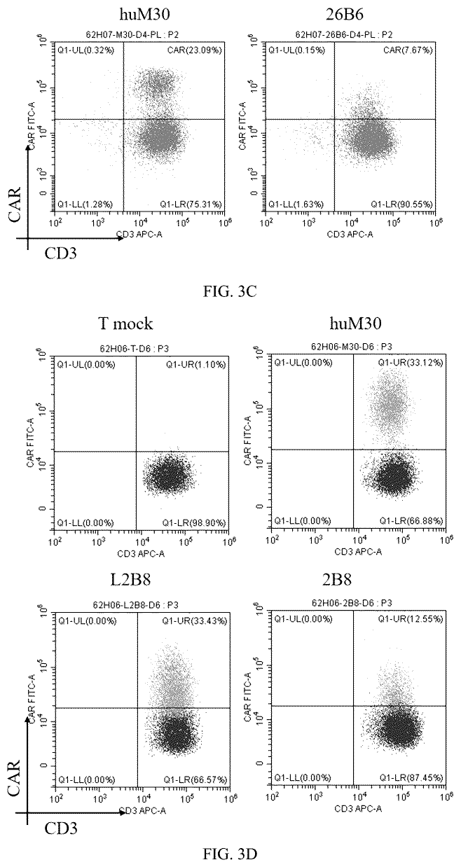

As shown in FIG. 3A, an expression rate of CAR in H26B6-CAR-T cells is 65.72%, and expression rates of CAR in H2B8-CAR-T, L2B8-CAR-T and L26B6-CAR-T cells are 31.73%, 38.15% and 44.14%, respectively.

As shown in FIG. 3B, an expression rate of CAR in Eno-CAR-T cells is 27.3%, and an expression rate of CAR in huM30-CAR-T cells is 45.2%.

In another experiment, as shown in FIG. 3C, an expression rate of CAR in huM30-CAR-T cells is 23.09%, and an expression rate of CAR in 26B6-CAR-T cells is 7.67%.

In another experiment, as shown in FIG. 3D, an expression rate of CAR in huM30-CAR-T cells is 33.12%, an expression rate of CAR in L2B8-CAR-T cells is 33.43%, and an expression rate of CAR in 2B8-CAR-T cells is 12.55%.

### Example 3 Killing Function of CAR-T Cells

H26B6-CAR-T, H2B8-CAR-T, L2B8-CAR-T and L26B6-CAR-T were co-incubated with human liver cancer cells HepG2, human pancreatic cancer cells PL45 and human cervical cancer cells SiHa for 16 h at effector to target ratios of 2:1, 1:1 and 1:4, and killing efficiency of CAR-T was detected using an RTCA technique.

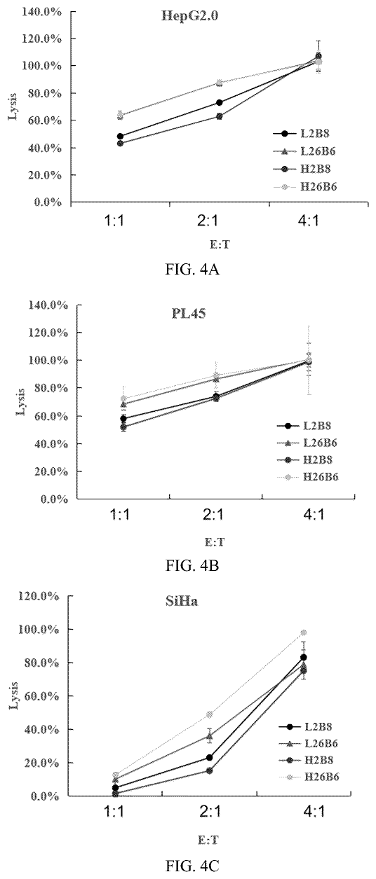

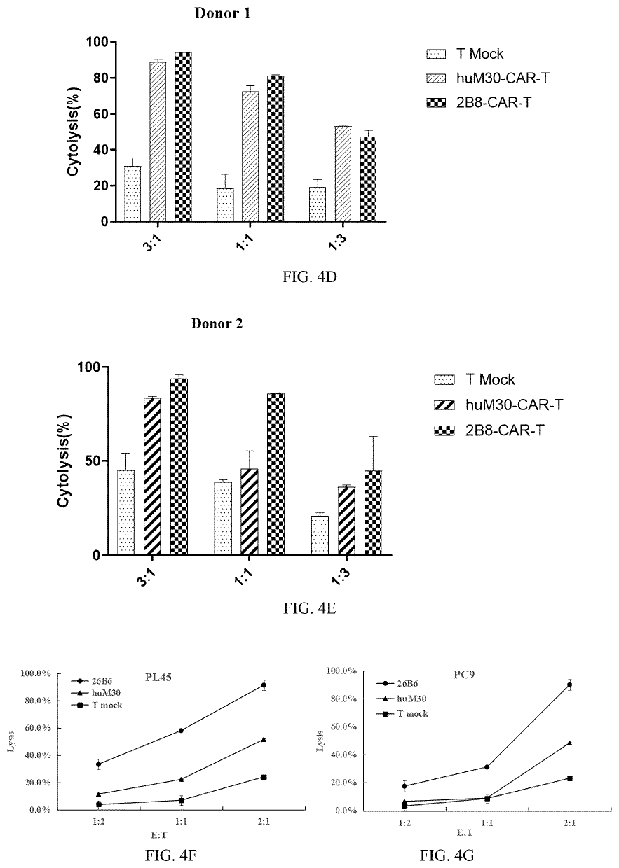

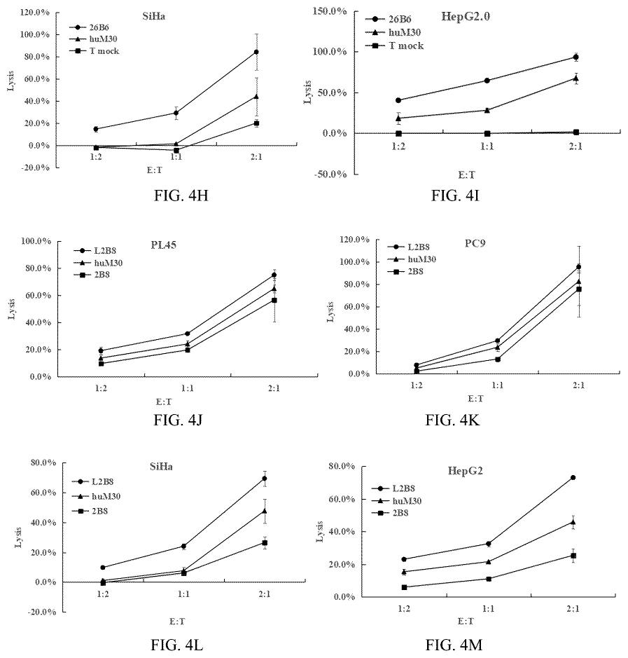

The results of FIGS. 4A, 4B and 4C show that the four types of CAR-T cells have killing effects on the three types of tumor cells at different effector to target ratios, and the greater the effector to target ratio is, the stronger the killing ability is; when the effector to target ratio is 2:1, the killing efficiency of H26B6-CAR-T on the three types of tumor cells is superior to that of H2B8-CAR-T, L2B8-CAR-T and L26B6-CAR-T.

Different CAR-T cells (huM30-CAR-T and 2B8-CAR-T) were prepared from PBMC of healthy donors (Donor 1 and Donor 2), respectively, and co-incubated with target cells at effector to target ratios of 3:1, 1:1 and 1:3 for 16 h. Killing efficiency of CAR-T was detected using the RTCA technique, and a T cell control group was set.

The results of FIGS. 4D and 4E show that huM30-CAR-T, 2B8-CAR-T and T cells have killing effects on the target cells at different effector to target ratios, where a killing ability of 2B8-CAR-T is significantly higher than those of huM30-CAR-T and the T cells at different effector to target ratios.

In another experiment, 26B6-CAR-T, huM30-CAR-T, L2B8-CAR-T, 2B8-CAR-T were co-incubated with human pancreatic cancer cells PL45, human lung cancer cells PC9, human cervical cancer cells SiHa and human liver cancer cells HepG2 for 16 h at effector to target ratios of 1:2, 1:1 and 2:1, and killing efficiency of CAR-T was detected using the RTCA technique.

As shown in FIGS. 4F, 4G, 4H and 4I, 26B6-CAR-T is superior to huM30-CAR-T in killing ability. As shown in FIGS. 4J, 4K, 4L and 4M, L2B8-CAR-T and 2B8-CAR-T are comparable to huM30-CAR-T in the ability to kill PL45 and PC9 cells, and L2B8-CAR-T is superior to huM30-CAR-T and 2B8-CAR-T in the ability to kill SiHa and HepG2.

### Example 4 Ability of CAR-T Cells to Secrete IFN-γ after Co-Culture With Tumor Cells

Eno-CAR-T cells were diluted with an RPMI-1640 serum-free medium containing 2 mM GlutaMAX, 10 mM HEPES, 100 U/mL penicillin and 100 μg/mL streptomycin and co-cultured with 1×10⁴ target cells (Daudi, H929, Jurkat, Tonly, A375, A549, PC9, HCT116, SY5Y, SH, MC or 293T), respectively, in a 96-well round bottom plate according to different effector to target ratios with three replicates disposed for each experiment and incubated for 16 h at 37° C. in a 5% CO₂ incubator. 50 μL supernatant was taken from each well to detect the secretion of cytokines IFN-γ.

huM30-CAR-T cells were diluted with an RPMI-1640 serum-free medium containing 2 mM GlutaMAX, 10 mM HEPES, 100 U/mL penicillin and 100 μg/mL streptomycin and co-cultured with 1×10⁴ target cells (Jurkat, A375, A549, HCT116, K562, SK-N-BE(2), HONE1 or HTB20), respectively, in a 96-well round bottom plate according to an effector to target ratio of 10:1 with three replicates disposed for each experiment and incubated for 16 h at 37° C. in a 5% CO₂ incubator. 50 μL supernatant was taken from each well to detect the secretion of cytokines IFN-γ.

A content of IFN-γ in the supernatant was detected using a human IFN-γ enzyme-linked immunosorbent kit (Shenzhen NeoBioscience Technology Co., Ltd): the supernatant was diluted 20 to 30-fold with a sample diluent in the kit, and 100 μL supernatant was drawn and added to a pre-coated ELISA plate and incubated for 1.5 h at 37° C. after sealing; after the incubated ELISA plate was washed with PBST and dried, 100 μL biotinylated antibody was added to each well, incubated for 1 h at 37° C., washed and dried; 100 μL HRP-labeled streptavidin was added to each well, wrapped with platinum paper, incubated in an incubator for 30 min at 37° C., washed and dried; 100 μL TMB substrate color developing liquid was added to each well, the reaction was conducted for 15 min at 37° C. in the dark, and 100 μL/well stopping solution was added to stop the reaction; an OD value at a wavelength of 450 nm was read with an Infinite F50 microplate reader (TECAN).

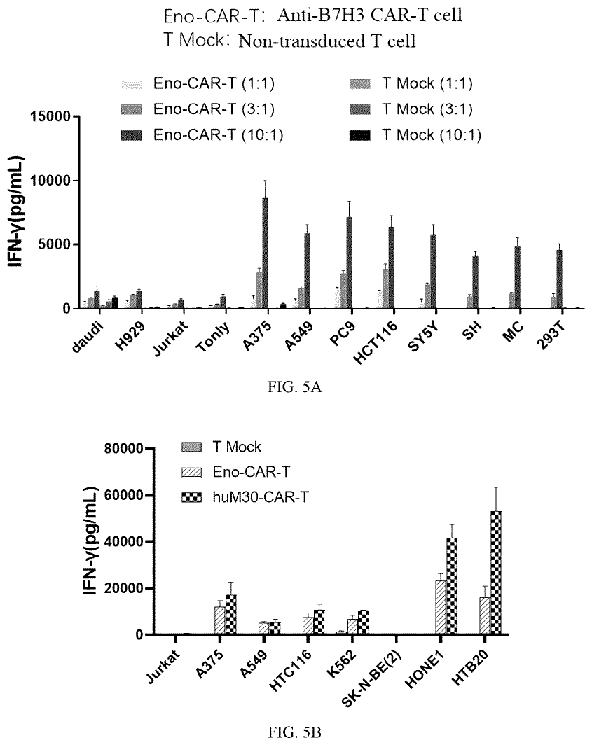

As shown in FIG. 5A, the Eno-CAR-T cells can release a large amount of IFN-γ when co-incubated with the B7H3-positive melanoma cells A375, the lung cancer cells A549 and PC9, the colon cancer cells HCT116 and the neuroblastoma SH-SYSY, SK-N-SH and SK-N-MC cells, but cannot release significant IFN-γ when co-incubated with the B7H3 expression-negative target cells Daudi, H929 and jurkat.

As shown in FIG. 5B, both huM30-CAR-T and Eno-CAR-T cells can kill A375, A549, HTC116, K562, HONE1 and HTB20, but have no killing effect on Jurkat and SK-N-BE(2).

### Example 5 In Vivo Pharmacodynamic Effect of H26B6-CAR-T

In this example, an in vivo pharmacodynamics effect of H26B6-CAR-T was further evaluated, NOD-Prkdcscid Il2rgtm1/Bcgen mice (B-NDG mice) were subcutaneously inoculated with human skin melanoma cells A375, human liver cancer cells Hep 3B2.1-7 or human cervical cancer cells SiHa to establish solid tumor models, and a growth inhibitory effect of H26B6-CAR-T on tumors in the mice was observed.

Steps are described below.

10 female B-NDG mice were selected and subcutaneously inoculated with A375-luc (luciferase-labeled human A375 cells; 5E+06/mouse; tumor formation for 5 days), 10 female B-NDG mice were selected and subcutaneously inoculated with Hep3B-luc (luciferase-labeled human Hep 3B2.1-7 cells; 5E+06/mouse; tumor formation for 9 days), and 25 female B-NDG mice were selected and subcutaneously inoculated with SiHa-luc (luciferase-labeled human SiHa cells; 5E+06/mouse; tumor formation for 9 days).

After tumors were formed, the mice were divided into groups according to an experimental plan, and the groups were injected with a vehicle (a DMSO injection), unmodified T cells (Mock T) and H26B6-CAR-T, respectively, with five mice in each group, where H26B6-CAR-T high, medium and low dose administration groups were set in a SiHa-luc group:
\textcircled{1} human skin melanoma A375 group: there were five mice in each group and two groups in total, where the two groups were administered Mock T 5×10⁶ cells/mouse and H26B6-CAR-T 3×10⁶ cells/mouse, respectively;
\textcircled{2} human liver cancer cells Hep 3B2.1-7 group: there were five mice in each group and two groups in total, where the two groups were administered Mock T 5×10⁶ cells/mouse and H26B6-CAR-T 3×10⁶ cells/mouse, respectively;
\textcircled{3} human cervical cancer SiHa group: there were five mice in each group and five groups in total, where one group was intravenously administered the vehicle (DMSO) 200 μL/mouse, one group was intravenously administered Mock T 5×10⁶ cell/mouse, the H26B6-CAR-T high dose administration group was intravenously administered 5×10⁶ cell/mouse, the H26B6-CAR-T medium dose administration group was intravenously administered 1×10⁶ cell/mouse, and the H26B6-CAR-T low dose administration group was intravenously administered 0.2×10⁶ cell/mouse.

The mice were subjected to clinical observation twice a day, weighed once before the grouping and twice a week after the administration. Sizes of the tumors were measured by a vernier caliper. The day of the administration of CAR-T was recorded as Day D0. Fluorescent signals were captured by a small animal living body imager. Blood was collected to detect contents of IFN-γ using ELISA.

No administration-related abnormality was seen in general clinical observation.

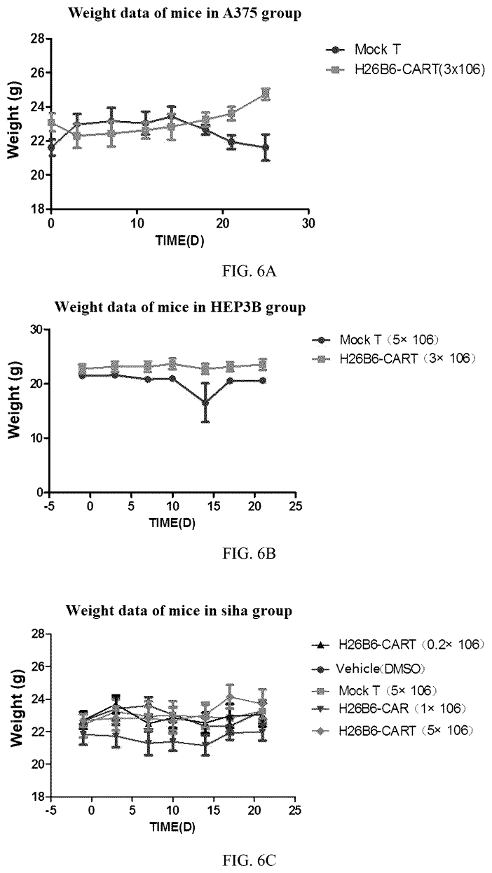

Variations of weights of animals in each group during the experiment are shown in FIGS. 6A, 6B and 6C, respectively. In the A375 group, the weight is stable in the first 15 days after the administration of Mock T and slightly decreased after 15 days, and the weight is stable and slightly increased after the administration of H26B6-CAR-T. In the Hep3B group, the weight is stable before and after the administration of Mock T and H26B6-CAR-T. In the SiHa group, the weight of mice is stable before and after the administration of Mock T and H26B6-CAR-T and no variation in statistical significance is seen.

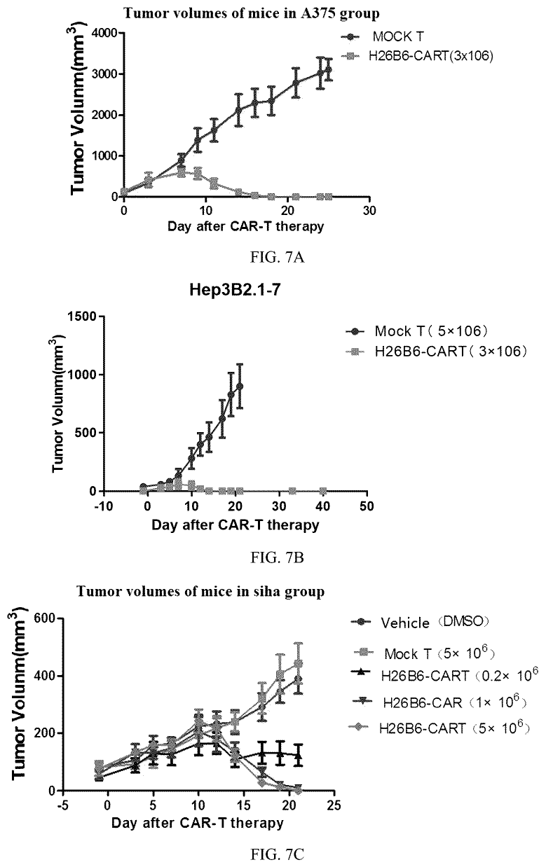

According to the tumor volume data in FIGS. 7A, 7B and 7C measured by the vernier caliper, in A375 and Hep3B tumor models, H26B6-CAR-T completely inhibits tumor growth; in a SiHa tumor model, high and medium doses of H26B6-CAR-T inhibit tumor growth so that the tumor is shrunk significantly, and the volume of the tumor treated with a low dose of H26B6-CAR-T is partially shrunk.

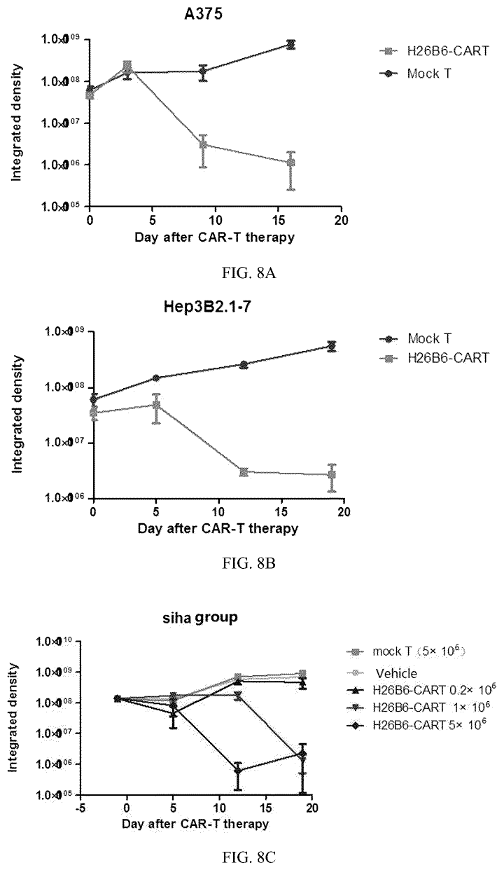

According to the intravital imaging fluorescence data in FIGS. 8A, 8B and 8C, in the A375 tumor model, a tumor inhibitory effect of H26B6-CAR-T relative to Mock T is significant on day 16 of the administration; in the Hep3B tumor model, a tumor inhibitory effect of H26B6-CAR-T relative to Mock T is significant on day 19 of the administration; in the SiHa tumor model, a tumor inhibitory effect of high and medium doses of H26B6-CAR-T relative to Mock T is significant on day 10 of the administration, and a low dose of H26B6-CAR-T has a partial inhibitory effect.

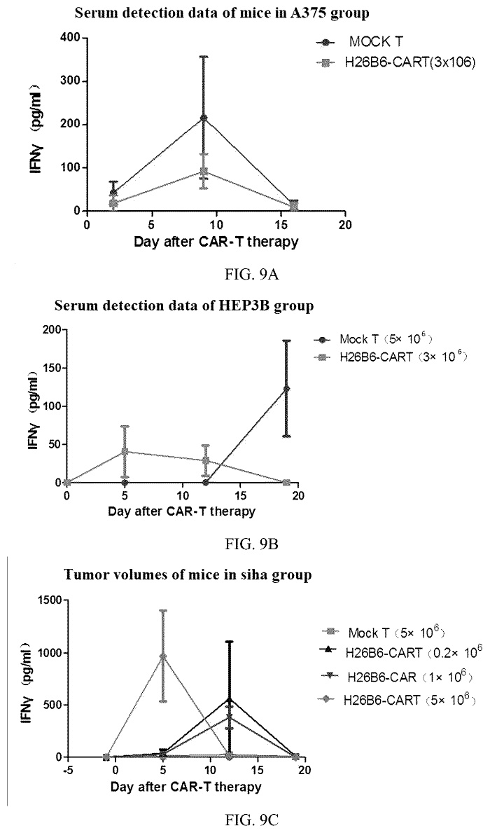

The secretion results of IFN-γ in serum detected through the ELISA are shown in FIGS. 9A, 9B and 9C. In the A375 tumor model, blood is collected to detect IFN-γ on D2, D9 and D16, where the secretion of IFN-γ is detected on D2 and gradually increased, and a secretion level of IFN-γ in the Mock T group is higher than that in the H26B6-CAR-T group. In the Hep3B tumor model, blood is collected to detect IFN-γ on D1, D5, D12 and D19, where the secretion of IFN-γ is detected in H26B6-CAR-T group on D5 and D12, and the secretion of IFN-γ is detected in Mock T group on D19. In the SiHa tumor model, blood is collected to detect IFN-γ on D1, D5, D12 and D19, where a peak value of the secretion of IFN-γ in the high dose H26B6-CAR-T group appears on day 5, and peak values of the secretion of IFN-γ in the low and medium dose H26B6-CAR-T groups and the Mock T group appear on day 12.

It indicates that H26B6-CAR-T can effectively eliminate the tumor cells in the three mice solid tumor models, tumors in the mice are significantly shrunk and no administration-related abnormality is seen.

In conclusion, the anti-B7H3 CAR-T cell of the present application has a significant killing effect on B7H3-positive tumor cells at different effector to target ratios and secretes a large number of cytokines IFN-γ after the co-culture with the tumor cells. The anti-B7H3 CAR-T cell has a significant in vivo pharmacodynamic effect and can effectively eliminate the B7H3-positive tumor cells.

The applicant has stated that although the detailed method of the present application is described through the examples described above, the present application is not limited to the detailed method described above, which means that the implementation of the present application does not necessarily depend on the detailed method described above. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. An anti-B7H3 chimeric antigen receptor, comprising an antigen-binding domain, a hinge region, a transmembrane domain and a signaling domain;
wherein the antigen-binding domain is an anti-B7H3 antibody.

2. The anti-B7H3 chimeric antigen receptor according to claim 1, wherein the antigen-binding domain comprises amino acid sequences shown in SEQ ID NO: 1 and SEQ ID NO: 2; or
the antigen-binding domain comprises amino acid sequences shown in SEQ ID NO: 3 and SEQ ID NO: 4; or
the antigen-binding domain comprises amino acid sequences shown in SEQ ID NO: 5 and SEQ ID NO: 6; or
the antigen-binding domain comprises amino acid sequences shown in SEQ ID NO: 7 and SEQ ID NO: 8; or
the antigen-binding domain comprises amino acid sequences shown in SEQ ID NO: 9 and SEQ ID NO: 10; or
the antigen-binding domain comprises amino acid sequences shown in SEQ ID NO: 11 and SEQ ID NO: 12; or
the antigen-binding domain comprises amino acid sequences shown in SEQ ID NO: 13 and SEQ ID NO: 14; or
the antigen-binding domain comprises amino acid sequences shown in SEQ ID NO: 15 and SEQ ID NO: 16.

3. The anti-B7H3 chimeric antigen receptor according to claim 1, wherein the hinge region comprises a CD8α hinge region.

4. The anti-B7H3 chimeric antigen receptor according to claim 1, wherein the transmembrane domain comprises a CD8α transmembrane region and/or a CD28 transmembrane region.

5. The anti-B7H3 chimeric antigen receptor according to claim 1, wherein the signaling domain comprises CD3ζ;
preferably, the signaling domain further comprises any one or a combination of at least two of 4-1BB, a CD28 intracellular region, DAP10 or OX40.

6. The anti-B7H3 chimeric antigen receptor according to claim 1, wherein the anti-B7H3 chimeric antigen receptor further comprises a signal peptide;
preferably, the signal peptide comprises any one of an IgGκ light chain signal peptide, a CD8α signal peptide, a GM-CSF signal peptide, an HSA signal peptide, an IgG heavy chain signal peptide, an IgG light chain signal peptide, a CD33 signal peptide, an IL-2 signal peptide or an insulin signal peptide.

7. The anti-B7H3 chimeric antigen receptor according to claim 1, wherein the anti-B7H3 chimeric antigen receptor comprises the signal peptide, the anti-B7H3 antibody, the CD8α hinge region, the CD8α transmembrane region, 4-1BB and CD3ζ.

8. The anti-B7H3 chimeric antigen receptor according to claim 1, wherein
the anti-B7H3 chimeric antigen receptor comprises an amino acid sequence shown in SEQ ID NO: 17; or
the anti-B7H3 chimeric antigen receptor comprises an amino acid sequence shown in SEQ ID NO: 18; or
the anti-B7H3 chimeric antigen receptor comprises an amino acid sequence shown in SEQ ID NO: 19; or
the anti-B7H3 chimeric antigen receptor comprises an amino acid sequence shown in SEQ ID NO: 20; or
the anti-B7H3 chimeric antigen receptor comprises an amino acid sequence shown in SEQ ID NO: 21; or
the anti-B7H3 chimeric antigen receptor comprises an amino acid sequence shown in SEQ ID NO: 22; or
the anti-B7H3 chimeric antigen receptor comprises an amino acid sequence shown in SEQ ID NO: 23; or
the anti-B7H3 chimeric antigen receptor comprises an amino acid sequence shown in SEQ ID NO: 24; or
the anti-B7H3 chimeric antigen receptor comprises an amino acid sequence shown in SEQ ID NO: 25; or
the anti-B7H3 chimeric antigen receptor comprises an amino acid sequence shown in SEQ ID NO: 26; or
the anti-B7H3 chimeric antigen receptor comprises an amino acid sequence shown in SEQ ID NO: 27; or
the anti-B7H3 chimeric antigen receptor comprises an amino acid sequence shown in SEQ ID NO: 28.

9. A nucleic acid molecule, comprising a coding gene of the anti-B7H3 chimeric antigen receptor according to claim 1.

10. The nucleic acid molecule according to claim 9, wherein
the nucleic acid molecule comprises a nucleic acid sequence shown in SEQ ID NO: 29; or
the nucleic acid molecule comprises a nucleic acid sequence shown in SEQ ID NO: 30; or
the nucleic acid molecule comprises a nucleic acid sequence shown in SEQ ID NO: 31; or
the nucleic acid molecule comprises a nucleic acid sequence shown in SEQ ID NO: 32; or
the nucleic acid molecule comprises a nucleic acid sequence shown in SEQ ID NO: 33; or
the nucleic acid molecule comprises a nucleic acid sequence shown in SEQ ID NO: 34; or
the nucleic acid molecule comprises a nucleic acid sequence shown in SEQ ID NO: 35; or
the nucleic acid molecule comprises a nucleic acid sequence shown in SEQ ID NO: 36; or
the nucleic acid molecule comprises a nucleic acid sequence shown in SEQ ID NO: 37; or
the nucleic acid molecule comprises a nucleic acid sequence shown in SEQ ID NO: 38; or
the nucleic acid molecule comprises a nucleic acid sequence shown in SEQ ID NO: 39; or
the nucleic acid molecule comprises a nucleic acid sequence shown in SEQ ID NO: 40.

11. An expression vector, comprising the nucleic acid molecule according to claim 9;
preferably, the expression vector is any one of a lentiviral vector, a retroviral vector or an adeno-associated viral vector containing the nucleic acid molecule according to claim 9, preferably the lentiviral vector.

12. A recombinant lentivirus prepared from a mammalian cell transfected with the expression vector according to claim 11 and a helper plasmid.

13. A chimeric antigen receptor T cell expressing the anti-B7H3 chimeric antigen receptor according to claim 1;
preferably, a genome of the chimeric antigen receptor T cell is integrated with a nucleic acid molecule; wherein the nucleic acid molecule comprises a coding gene of the anti-B7H3 chimeric antigen receptor according to claim 1;
preferably, the chimeric antigen receptor T cell comprises an expression vector and/or a recombinant lentivirus;
wherein the expression vector comprises the nucleic acid molecule, and the recombinant lentivirus is prepared from a mammalian cell transfected with the expression vector and a helper plasmid.

14. A pharmaceutical composition, comprising the chimeric antigen receptor T cell according to claim 13;
optionally, the pharmaceutical composition further comprises any one or a combination of at least two of a pharmaceutically acceptable carrier, excipient or diluent.

15. (canceled)

16. A method for treating a malignant tumor, comprising administering an effective amount of the anti-B7H3 chimeric antigen receptor according to claim 1 to subject in need thereof;
preferably, the malignant tumor comprises any one or a combination of at least two of acute lymphoblastic leukemia, myeloid leukemia, melanoma, neuroblastoma, non-small-cell lung cancer, nasopharyngeal carcinoma, breast cancer, colorectal cancer, liver cancer, pancreatic cancer or cervical cancer.
